(19) United States
(12) Patent Application Publication
(10) Pub. No.: US 2025/0250623 A1
(43) Pub. Date: Aug. 7, 2025

(54) ULTRA-FAST ONE-POT EXPONENTIAL ISOTHERMAL AMPLIFICATION OF NUCLEIC ACIDS

(71) Applicant: DOMUS DIAGNOSTICS, INC., Park City, UT (US)

(72) Inventors: Xin SONG, Raleigh, NC (US); John H. REIF, Durham, NC (US)

(21) Appl. No.: 19/047,589

(22) Filed: Feb. 6, 2025

Related U.S. Application Data
(60) Provisional application No. 63/550,424, filed on Feb. 6, 2024.

(51) Int. Cl. C12Q 1/6844

(52) U.S. Cl. CPC C12Q 1/6844

(57) ABSTRACT

Disclosed are assays for ultra-fast exponential isothermal amplification of nucleic acids. The assays can improve target detection specificity by using three adjacent binding sites on the target sequence, compared to just one trigger sequence in conventional EXPAR, to initiate the exponential amplification reaction. Amplification of an EXPAR trigger sequence can be carried out via a two-stage process, wherein a first stage achieves linear amplification of the trigger sequence upon successful detection of three adjacent binding sites specified on the target nucleic acid, followed by a second stage that achieves subsequent exponential amplification.

### CROSS-REFERENCE TO RELATED APPLICATIONS

This application claims priority to and the benefit of U.S. Provisional Patent Application No. 63/550,424, filed Feb. 6, 2024, the entirety of which is incorporated herein by reference.

### BACKGROUND

Improving the turnaround time (TAT) of molecular diagnostic testing is pivotal for rapid and accurate identification of pathogens, initiating timely treatments, and enhancing public health responses to control outbreaks and safeguard communities. In infectious disease scenarios such as with COVID-19, quick identification aids in isolation measures, contact tracing, and containment efforts. Molecular tests such as PCR and RT-PCR are typically performed at central laboratories requiring expensive equipment, sophisticated protocol, and trained personnel, generally taking two to three days to get a result after the sample is received by the lab.

Various techniques of isothermal nucleic acid amplification¹ have been developed and commercialized to enable simpler and affordable molecular testing at the point of care and at home. For example, loop-mediated isothermal amplification (LAMP)² is a widely used technique with, however, a typical TAT of 30 minutes to 1 hour from sample to result.

Accordingly, there is an ongoing need for compositions and methods capable of providing effective diagnostic testing with short TAT.

### SUMMARY

The present disclosure is directed to assays (including compositions and methods) for ultra-fast exponential isothermal amplification that can provide sensitive and specific detection of nucleic acids. Compared to conventional EXPAR assays, the disclosed embodiments can address limitations of EXPAR and can facilitate enhancements including, for example: (1) improving target detection specificity by using three adjacent binding sites on the target sequence (compared to just one trigger sequence in conventional EXPAR) to initiate the exponential amplification reaction; (2) The embodiments can detect DNA and/or RNA targets without the need for modifying the target transduction mechanism. Notably, for RNA detection, the disclosed assays can eliminate the need for reverse transcription of lengthy nucleotide sequences, thereby minimizing the rate-limiting impact of the reverse transcription step on the overall assay. This can lead to improved reaction speed and sensitivity. (3) The designs can enable ultra-fast isothermal nucleic acid amplification assays in a convenient one-pot format such as in solution, air-dried, or freeze-dried format, (4) The amplification of an EXPAR trigger sequence can be carried out via a two-stage process, wherein the first stage achieves linear amplification of the trigger sequence generated upon successful detection of three adjacent binding sites specified on the target nucleic acid, followed by the second stage that achieves a subsequent exponential amplification (or linear amplification in some embodiments) triggered by a successful first stage. Such reaction schemes can improve the sensitivity and detection limit of the assays. (5) Certain primers used in the disclosed assays (e.g., Primer A, Templated Primer A, Gated Primer A, Ungated Primer A) include both a target-specific binding domain and domains with EXPAR-relevant functionality (e.g., EXPAR trigger sequence, nicking enzyme recognition site sequence, etc.), the latter of which are independent of the target nucleic acid sequence and its binding sites. This can effectively decouple the selection of the primer binding sites from the design of sequences involved in the downstream EXPAR amplification, enabling simple and flexible assay designs for specific detection of different DNA and RNA targets. (6) A primer comprising both a target-specific binding domain and domains with EXPAR-relevant functionality can be adapted to utilize variations of the disclosed designs to enable additional assay features or improve the assay performance. For example, an assay can include multiple such primers (e.g., Primer A, Templated Primer A, Gated Primer A, Ungated Primer A) designed to have the same EXPAR trigger domain but incorporate different target-specific binding domains to simultaneously detect multiple different targets, such as multiple different target regions on a target genome. This enables the generation of multiple linear amplifiers of the same EXPAR trigger upon detection of each instance of the target nucleic acid sequence within the reaction, which can further improve the sensitivity and speed of the assay. (7) The disclosed assays can incorporate one of more mechanisms to suppress non-specific amplification and/or improve the amplification efficiency, including utilizing a hairpin stem-loop structure at the 5′ end of the template to reduce or eliminate the unproductive binding of the trigger sequence at the 5′ end of the template. (8) The disclosed target transduction mechanism (i.e., converting the detection of the target DNA or RNA sequence into amplification of an EXPAR trigger sequence) described herein synergizes with universal LAMP assays that previously introduced (U.S. patent application Ser. No. 17/749,858). This permits the application of conventional LAMP primer design principles and software to choose suitable primer binding sites on the target nucleic acid sequences to aid in the design of target-specific primers for implementing the ultra-fast one-pot exponential isothermal amplification assays described herein. (9) The present disclosure also describes assays enabling one-pot multiplexed detection of different targets and the utilization of molecular logic computation to improve the assay specificity and multiplexity.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification.

FIG. 1 illustrates the components of an ultra-fast one-pot isothermal nucleic acid amplification reaction. Reaction buffers and salts are not depicted.

FIG. 2 illustrates the mechanism of an ultra-fast one-pot isothermal nucleic acid amplification reaction based on components shown in FIG. 1.

FIG. 3 illustrates the components of another design of an ultra-fast one-pot isothermal nucleic acid amplification reaction. Reaction buffers and salts are not depicted. This design features the use of a templated primer, eliminating the need for an EXPAR template.

FIG. 4 illustrates the mechanism of an ultra-fast one-pot isothermal nucleic acid amplification reaction based on components shown in FIG. 3.

FIG. 5 illustrates the components of another design of an ultra-fast one-pot isothermal nucleic acid amplification reaction. Reaction buffers and salts are not depicted. This design features the use of a gated primer. The gated primer can be pre-annealed and purified.

FIG. 6 illustrates the mechanism of an ultra-fast one-pot isothermal nucleic acid amplification reaction based on components shown in FIG. 5.

FIG. 7 illustrates the components of another design of an ultra-fast one-pot isothermal nucleic acid amplification reaction. Reaction buffers and salts are not depicted. This design features the use of a single-stranded ungated primer which reacts with a single-stranded initiator to form a gated primer required for the amplification reaction. This design eliminates the need to pre-anneal and purify the gated primer.

FIG. 8 illustrates the mechanism of an ultra-fast one-pot isothermal nucleic acid amplification reaction based on components shown in FIG. 7.

FIG. 9 illustrates example designs of the EXPAR template incorporating mechanisms to improve the amplification efficiency of EXPAR reactions. One of more of these designs can be applied to enhance the different designs of the ultra-fast one-pot isothermal nucleic acid amplification reactions described in the present disclosure.

FIG. 10 illustrates the mechanism of the improved isothermal nucleic acid amplification reaction based on one of the enhanced EXPAR template designs illustrated in FIG. 9.

FIGS. 11A-11D illustrate the components of an ultra-fast one-pot isothermal nucleic acid amplification reaction featuring different designs of a molecular logic AND Gate to improve the target detection specificity and multiplexity. Reaction buffers and salts are not depicted.

FIGS. 12A and 12B illustrate the mechanism governing the initial phase of an ultra-fast one-pot isothermal nucleic acid amplification reaction based on components shown in FIG. 11. FIG. 12A illustrates the mechanism of generating a linearly amplified EXPAR trigger specific to the detection of the 1^{st} target RNA or DNA. FIG. 12B illustrates the mechanism of generating a linearly amplified EXPAR trigger specific to the detection of the 2^{nd} target RNA or DNA.

FIGS. 13A-13D illustrate the mechanism governing the second phase of an ultra-fast one-pot isothermal nucleic acid amplification reaction based on components shown in FIG. 11. FIG. 13A illustrates the mechanism of exponentially amplifying the EXPAR Trigger X based on the AND Gate Design variation 1 illustrated in FIG. 11 following linear amplification of both EXPAR Trigger X and Trigger Y from FIG. 12. FIG. 13B illustrates the mechanism of exponentially amplifying the EXPAR Trigger Y based on the AND Gate Design variation 2 illustrated in FIG. 11 following linear amplification of both EXPAR Trigger X and Trigger Y from FIG. 12. FIGS. 13C and 13D illustrate the mechanism of linearly amplifying an EXPAR Trigger Z based on the AND Gate Design variation 3 and 4, respectively, illustrated in FIG. 11 following linear amplification of both EXPAR Trigger X and Trigger Y from FIG. 12.

FIG. 14 illustrates the design and mechanism of exponential cross-amplification reaction initiated by the presence of one or both types of the EXPAR triggers linearly amplified from the specific detection of the 1^{st} and the 2^{nd} RNA or DNA targets, respectively. This reaction essentially computes a logic OR between detection of the 1^{st} and the 2^{nd} RNA or DNA target. This design eliminates the use of duplicate domains on the EXPAR templates, offering advantages in mitigating the common occurrence of non-specific amplifications in EXPAR reactions.

FIG. 15 illustrates example designs and mechanisms of multiplexed detection of different targets based on an ultra-fast one-pot isothermal nucleic acid amplification reaction combined with molecular beacons that react specifically to different EXPAR triggers amplified from the detection of different RNA and/or DNA targets. Multiplexed readout can be achieved by fluorescence-generating mechanisms other than molecular beacons. Examples include but are not limited to Scorpions probes, and strand-displacement probes such as assimilating probes, detection of amplification by releasing of quenching (DARQ) probes, oligonucleotide strand exchange (OSD) probes, and the like.

### DETAILED DESCRIPTION

### Introduction

The compositions and methods described herein can be utilized in conjunction with any of the compositions, devices, and/or methods disclosed in U.S. patent application Ser. No. 17/749,858, filed May 20, 2022; Ser. No. 18/205,992, filed Jun. 5, 2023; Ser. No. 18/370,117, filed Sep. 19, 2023; Ser. No. 18/551,547, filed Sep. 20, 2023; Ser. No. 18/752,268, filed Jun. 24, 2024; Ser. No. 18/970,704, filed Dec. 5, 2024; Ser. No. 19/011,449, filed Jan. 6, 2025; and 63/660,882, filed Jun. 17, 2024. Each of the foregoing applications is incorporated herein by reference.

To improve the speed of molecular testing, the inventors developed methods and compositions of ultra-fast one-pot loop-mediated isothermal amplification assays for detection of DNA and RNA (U.S. application Ser. No. 19/011,449) that enable sensitive molecular diagnostics within minutes. Nucleic acid tests designed to provide fast amplification include recombinase polymerase amplification (RPA)³ (see U.S. Pat. No. 7,666,598), nicking enzyme amplification reaction (also known as nicking and extension amplification reaction or NEAR; see US Patent App. No. US2009/0081670), and variations of these techniques. Such techniques are designed to achieve 8-15 min TAT, but issues relating to sensitivity and specificity are common.

Exponential amplification reaction^{4,5} (known as EXPAR) is another type of rapid isothermal amplification technique utilizing nicking enzyme and polymerase to exponentially amplify a short trigger sequence based on repeated nicking and strand displacement polymerization reactions. Despite extensive research and various proposed strategies aimed at improving its design,⁶⁻¹⁰ EXPAR is still challenged by issues including non-specific amplification. Furthermore, there is a lack of universally applicable and straightforward methods for reliably detecting arbitrary sequences of RNA and DNA using EXPAR. Existing methods for generating the trigger sequences needed for the exponential amplification in EXPAR tend to be complex and application specific.¹¹⁻¹⁵

In the drawings of the present disclosure, DNA and RNA strands are abstracted into functional domains,¹⁹ which represent consecutive nucleotide bases that function as a unit in hybridization and dissociation of oligonucleotide molecules. Unless otherwise specified, such domains are represented by thick lines and are labeled with letters and/or letter-number combinations in the drawings. Thin lines connecting domains on a DNA or RNA strand represent continuous nucleotide bases that are not involved at the domain hybridization/dissociation site. The apostrophe notation added to a domain name represents the reverse complementary sequence of the domain. The direction of nucleic acid sequences is indicated by an arrow pointing in the 5′→3′ direction. For example, a target genomic sequence may have 3 adjacent binding sites on its sequence represented as domains P3, P1, and P2 arranged in the 5′→3′ direction, and a primer designed with a domain P1′ on its 3′-end can hybridize to the P1 domain on the target sequence due to sequence complementarity.

The present disclosure refers to the various components (e.g., primers, domains, templates) of the compositions and methods using generic representative labels as discussed above. The skilled person is readily capable of applying such generic labels to specific target sequences without undue experimentation. That is, the skilled person is readily capable of selecting a suitable target sequence and then designing primers that meet the requirements of the disclosed embodiments in relation to the selected target sequence. For example, once a target sequence is identified, the skilled person can define adjacent primer binding sites on the target sequence (e.g., P3, P1, P2 along the 5′→3′ direction) and then design the primers and other disclosed components accordingly based on disclosed relationships to those domains (e.g., the P3 and reverse complementary sequences P1′ and P2′ used in the primers). The skilled person is also capable of selecting suitable sequences to function as nicking enzyme recognition sites and EXPAR trigger domains. The skilled person is also capable of configuring standard isothermal amplification parameters such as determining suitable temperature ranges and selecting suitable solution components (e.g., buffers, salts, molecular enhancers, excipients, and/or fluorescent or colorimetric indicators such as DNA intercalating dyes for visualization of the amplified reaction products) to effectively create and carry out the disclosed embodiments.

### Example Compositions & Methods

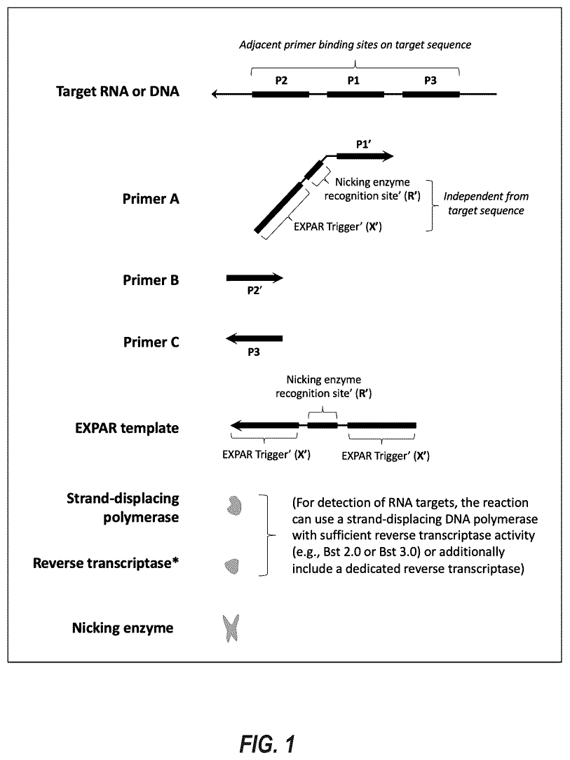

FIG. 1 illustrates the components of an ultra-fast one-pot isothermal nucleic acid amplification reaction. Components for such a reaction include (1) a Target RNA or DNA sequence, that contains subsequences P3, P1, and P2 (listed in the 5′→3′ direction) as three adjacent primer binding sites; (2) a Primer A, that is a primer nucleic acid strand containing subsequences forming an EXPAR Trigger′ (X′) domain, a Nicking enzyme recognition site′ (R′) domain, and a P1′ domain, respectively, listed in the 5′→3′ direction. The X′ domain is located at the 5′ end of the Primer A, and the P1′ domain is located at the 3′ end of the Primer A. The nucleic acid sequences of the X′ domain and the R′ domain are independent of the Target RNA or DNA sequence; (3) a Primer B, that is a primer nucleic acid strand with sequence P2′; (4) a Primer C, that is a primer nucleic acid strand with sequence P3; (5) an EXPAR template, that contains subsequences forming an EXPAR Trigger′ (X′) domain, a Nicking enzyme recognition site′ (R′) domain, and an EXPAR Trigger′ (X′) domain, respectively, listed in the 5′→3′ direction; (6) a strand-displacing polymerase. For detection of RNA targets, the reaction can use a strand-displacing DNA polymerase with sufficient reverse transcriptase activity (e.g., Bst 2.0 or Bst 3.0) or additionally include (7) a dedicated reverse transcriptase; (8) a nicking enzyme (e.g., Nt.BstNBI) that nicks one strand of a double-stranded DNA at the specific cut site recognized by the nicking enzyme.

Standard nucleic acid reaction components such as reaction buffers and salts are not depicted. Similarly, optional additives such as molecular enhancers, excipients, duplex DNA intercalating dyes (e.g., SYTO 9, EvaGreen, EvaGreen Plus, dsGreen) or other fluorescent or colorimetric indicators for visualization of the amplified reaction products are not depicted in this and the following drawings.

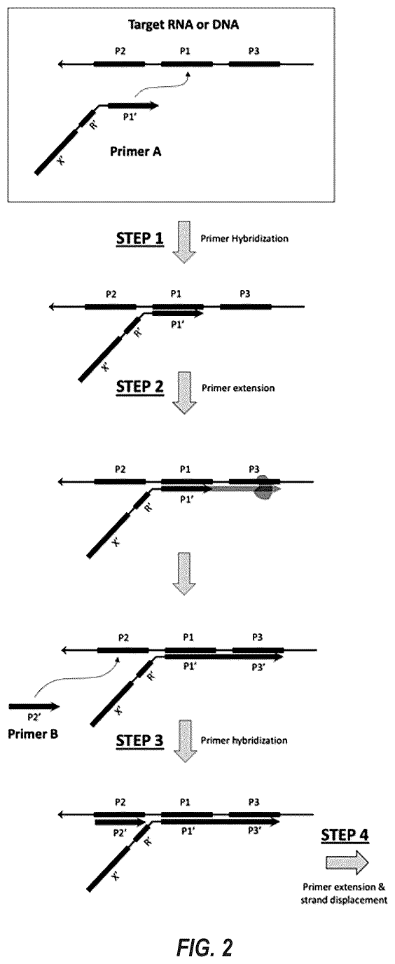

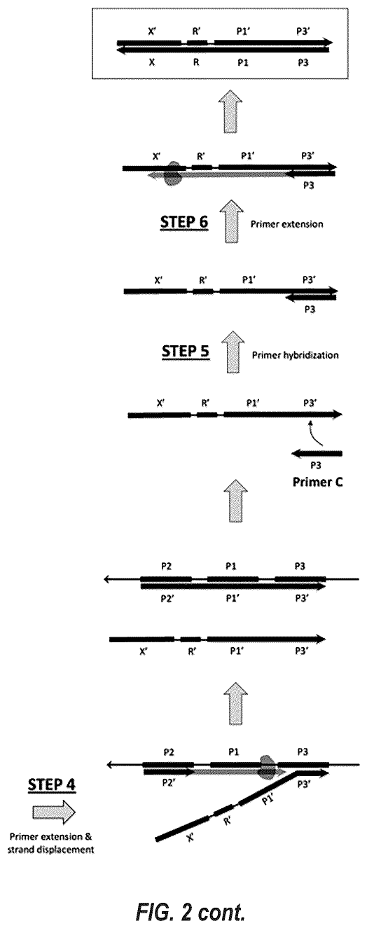

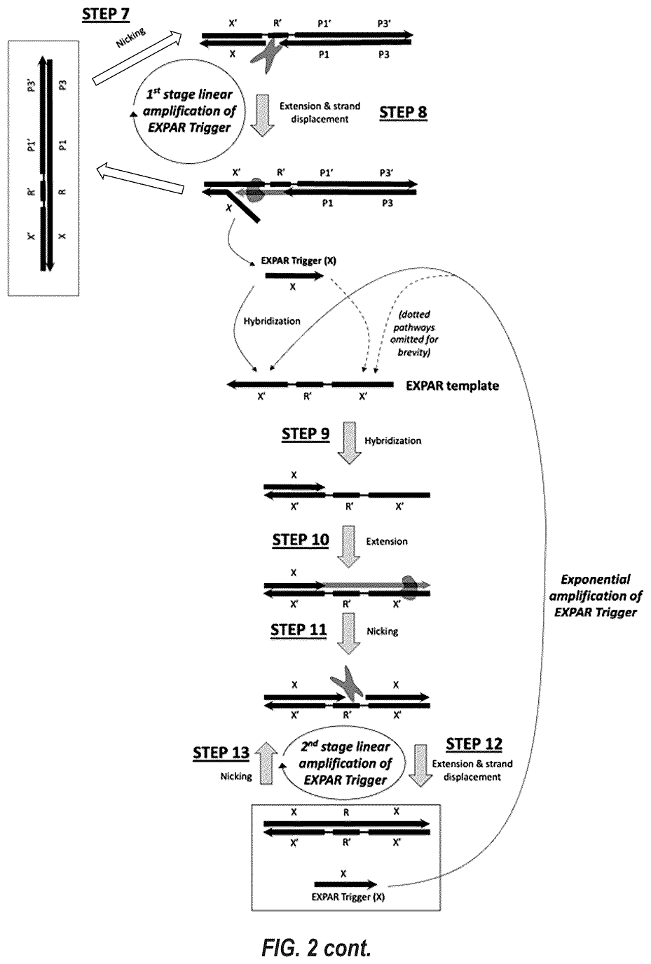

FIG. 2 illustrates the mechanism of an ultra-fast one-pot isothermal nucleic acid amplification reaction based on components listed in FIG. 1. The reaction contains two stages. The first stage is considered as a target transduction stage which converts the detection of a target RNA or DNA strand into linear amplification of a Trigger strand, which is then exponentially amplified in the second stage by an EXPAR reaction. Specifically, in STEP 1, the Primer A hybridizes to the target RNA or DNA strand by binding to the P1 domain. In STEP 2, the 3′ end of the Primer A is extended by polymerase forming a domain P3′ that is complementary to the domain P3 on the target sequence. In STEP 3, the Primer B hybridizes to the target RNA or DNA strand by binding to the P2 domain. In STEP 4, the 3′ end of the Primer B is extended by polymerase, and via primer extension and strand displacement, displacing the extended Primer A (i.e., Primer A with the extended P3′ domain) from the duplex DNA. This results in a single-stranded sequence that contains domains X′, R′, P1′, and P3′ listed in the 5′→3′ direction. There may be additional bases attached to the 3′ end of the P3′ domain due to further extension by polymerase and for brevity, those bases are not depicted in the drawing. In STEP 5, the Primer C hybridizes to the single-stranded DNA generated from STEP 4 by binding to the P3′ domain. In STEP 6, the 3′ end of the Primer C is extended by polymerase forming a double-stranded DNA containing domains P3, P1, R, and X in the bottom strand listed in the 5′→3′ direction. In STEP 7, the nicking enzyme recognizes the R′/R domain and nicks the bottom strand. In STEP 8, the polymerase extends the 3′ end of the strand at the nicking site and releases the nucleic acid sequence X by strand displacement and reforms the double-stranded DNA. This duplex DNA serves as a template for repeated nicking, polymerase extension, and displacement reactions, linearly amplifying the nucleic acid sequence X that functions as EXPAR Trigger (X) for next step. In STEP 9, the EXPAR Trigger (X) hybridizes to the EXPAR template at the X′ domains. For brevity, the drawing illustrates only the productive reaction pathways corresponding to EXPAR Trigger hybridization at the 3′ end of the EXPAR template because EXPAR Trigger hybridized at the 5′ end of the EXPAR Template does not trigger effective amplification and will be displaced by extension of the EXPAR Trigger hybridized at the 3′ end of the EXPAR Template. In STEP 10, the 3′ end of the EXPAR Trigger X is extended by polymerase forming a double-stranded DNA containing domains X, R, and X in the top strand listed in the 5′→3′ direction. In STEP 11, the nicking enzyme recognizes the R′/R domain and nicks the top strand. Via repeated nicking, extension, and strand displacement reactions illustrated in STEP 12 and 13, the EXPAR Trigger (X) is linearly amplified. Newly generated EXPAR Trigger (X) can hybridize to more single-stranded EXPAR templates in the reaction, forming duplex DNA complexes that each serves as a linear amplifier of the EXPAR Trigger (X). As a result, this creates a rapid, exponential amplification of the Trigger (X).

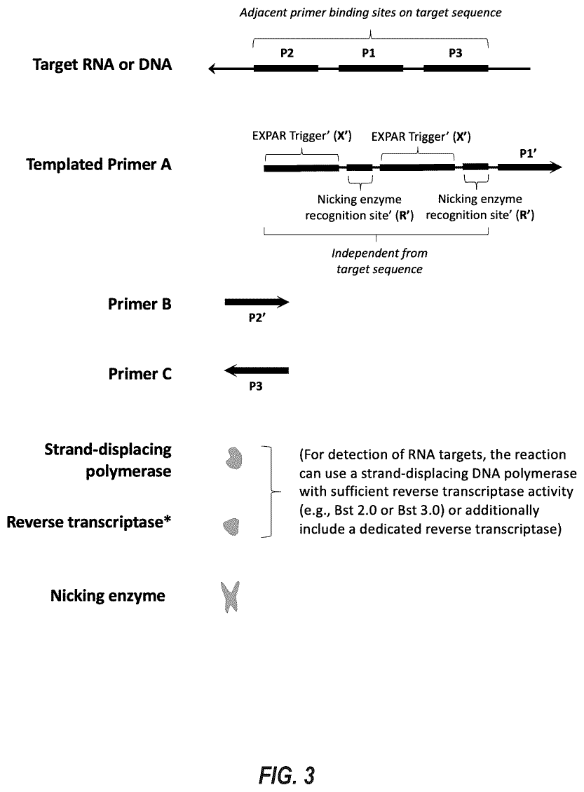

FIG. 3 illustrates the components of another design of an ultra-fast one-pot isothermal nucleic acid amplification reaction. Compared to the design illustrated in FIG. 1 and FIG. 2, this design eliminates the requirement of an EXPAR Template as its function is provided by the Templated Primer A. Main components for such a reaction include (1) a Target RNA or DNA sequence, that contains subsequences P3, P1, and P2 (listed in the 5′→3′ direction) as three adjacent primer binding sites; (2) a Templated Primer A, that is a primer nucleic acid strand containing subsequences forming an EXPAR Trigger′ (X′) domain, a Nicking enzyme recognition site′ (R′) domain, an EXPAR Trigger′ (X′) domain, a Nicking enzyme recognition site′ (R′) domain, and a P1′ domain, respectively, listed in the 5′→3′ direction. The P1′ domain is located at the 3′ end of the Templated Primer A. The nucleic acid sequences of the X′ domains and the R′ domains are independent of the Target RNA or DNA sequence; (3) a Primer B, that is a primer nucleic acid strand with sequence P2′; (4) a Primer C, that is a primer nucleic acid strand with sequence P3; (5) a strand-displacing polymerase. For detection of RNA targets, the reaction can use a strand-displacing DNA polymerase with sufficient reverse transcriptase activity (e.g., Bst 2.0 or Bst 3.0) or additionally include (6) a dedicated reverse transcriptase; (7) a nicking enzyme (e.g., Nt.BstNBI) that nicks one strand of a double-stranded DNA at the specific cut site recognized by the nicking enzyme. As mentioned previously, standard nucleic acid reaction components such as reaction buffers, salts, and other additives such as molecular enhancers, excipients, and fluorescent or colorimetric indicators are not depicted in this and the following drawings.

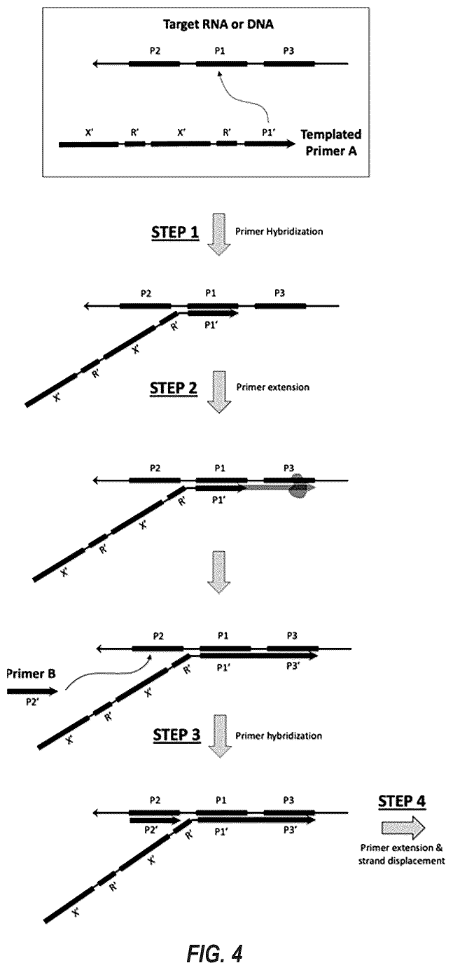

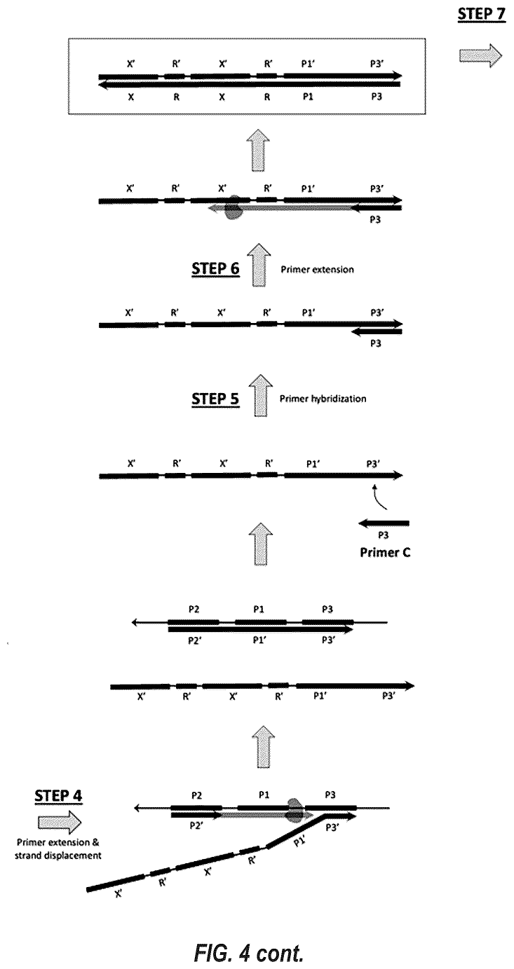

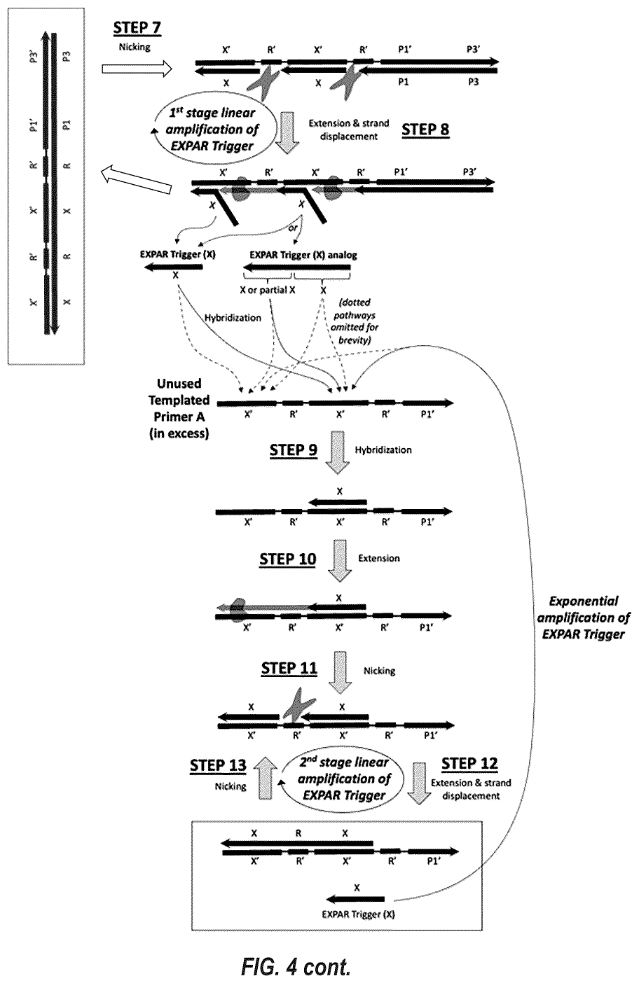

FIG. 4 illustrates the mechanism of an ultra-fast one-pot isothermal nucleic acid amplification reaction based on components listed in FIG. 3. The reaction contains two stages. The first stage is considered as a target transduction stage which converts the detection of a target RNA or DNA strand into linear amplification of a Trigger strand, which is then exponentially amplified in the second stage by an EXPAR reaction. Specifically, in STEP 1, the Templated Primer A hybridizes to the target RNA or DNA strand by binding to the P1 domain. In STEP 2, the 3′ end of the Templated Primer A is extended by polymerase forming a domain P3′ that is complementary to the domain P3 on the target sequence. In STEP 3, the Primer B hybridizes to the target RNA or DNA strand by binding to the P2 domain. In STEP 4, the 3′ end of the Primer B is extended by polymerase, and via primer extension and strand displacement, displacing the extended Templated Primer A (i.e., Templated Primer A with the extended P3′ domain) from the duplex DNA. This results in a single-stranded sequence that contains domains X′, R′, X′, R′, P1′, and P3′ listed in the 5′→3′ direction. There may be additional bases attached to the 3′ of the P3′ domain due to further extension by polymerase and for brevity, those bases are not depicted in the drawing. In STEP 5, the Primer C hybridizes to the single-stranded DNA generated from STEP 4 by binding to the P3′ domain. In STEP 6, the 3′ end of the Primer C is extended by polymerase forming a double-stranded DNA containing domains P3, P1, R, X, R, and X in the bottom strand listed in the 5′→3′ direction. In STEP 7, the nicking enzyme recognizes the R′/R domains and nicks the bottom strand. In STEP 8, the polymerase extends the 3′ ends of the strands at both nicking sites, and via strand displacement, releases the EXPAR Trigger X that is a nucleic acid sequence X and the EXPAR Trigger (X) analog that contains either a complete or partial subsequence X at its 3′ end. This process also reforms the double-stranded DNA which serves as a template for repeated nicking, polymerase extension, and displacement reactions, linearly amplifying both the EXPAR Trigger (X) and the EXPAR Trigger (X) analog for the next step. In STEP 9, the EXPAR Trigger (X) and the EXPAR Trigger (X) analog hybridize to the EXPAR template at the X′ domains. For brevity, the drawing illustrates only the productive reaction pathways corresponding to EXPAR Trigger hybridization closer to the 3′ end of the EXPAR template because EXPAR Trigger hybridized at the 5′ end of the EXPAR Template does not trigger effective amplification. Similarly, the pathways corresponding to hybridization between the X domain at 5′ end of the EXPAR Trigger (X) analog and the EXPAR template are omitted. In STEP 10, the 3′ end of the EXPAR Trigger X (or EXPAR Trigger X analog) is extended by polymerase forming a double-stranded region containing domains X, R, and X in the top strand listed in the 5′→3′ direction. In STEP 11, the nicking enzyme recognizes the R′/R domain and nicks the top strand. Via repeated nicking, extension, and strand displacement reactions illustrated in STEP 12 and 13, the EXPAR Trigger (X) is linearly amplified. Newly generated EXPAR Trigger (X) can hybridize to more single-stranded Templated Primer A in the reaction, forming duplex DNA complexes that each serves as a linear amplifier of the EXPAR Trigger (X). As a result, this creates a rapid, exponential amplification of the Trigger (X).

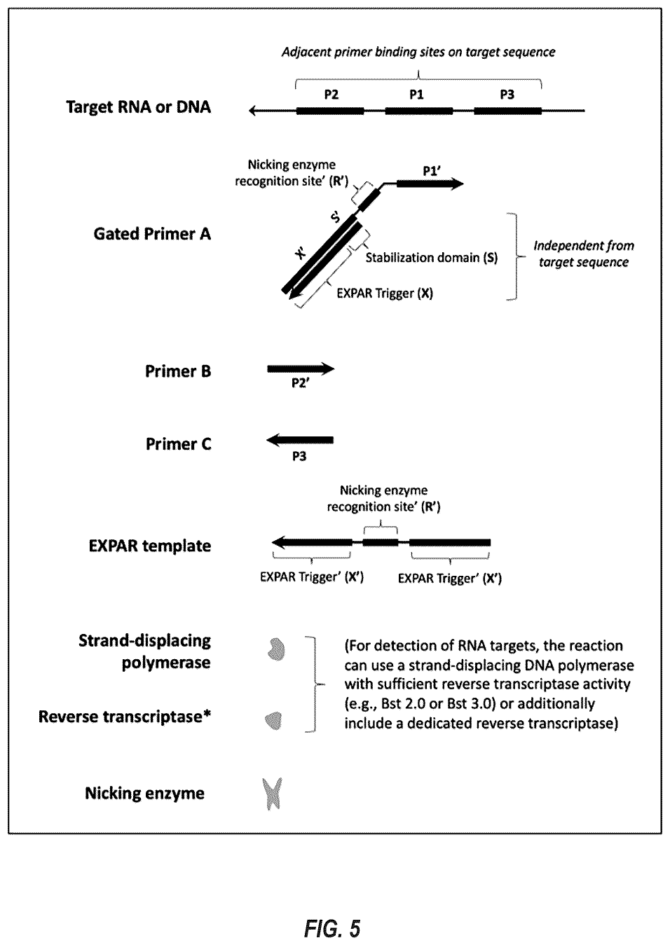

FIG. 5 illustrates the components of another design of an ultra-fast one-pot isothermal nucleic acid amplification reaction. Reaction buffers and salts are not depicted. Compared to the design illustrated in FIG. 1 and FIG. 2, this design uses a pre-annealed Gated Primer A instead of a single-stranded Primer A. The Gated Primer A features a double-stranded stabilization domain that prevents unintended dehybridization of the EXPAR Trigger (X) at the reaction temperature. Main components for such a reaction include (1) a Target RNA or DNA sequence, that contains subsequences P3, P1, and P2 (listed in the 5′→3′ direction) as three adjacent primer binding sites; (2) a Gated Primer A, that is a pre-annealed nucleic acid complex formed by a single-stranded DNA containing subsequences forming a Stabilization domain(S) and an EXPAR Trigger (X) domain (listed in the 5′→3′ direction) hybridized to a single-stranded DNA containing subsequences forming an EXPAR Trigger′ (X′) domain, an S′ domain that is reverse complementary to the S domain, a Nicking enzyme recognition site′ (R′) domain, and a P1′ domain, respectively, listed in the 5′→3′ direction. The X′ domain is located at the 5′ end of the Gated Primer A, and the P1′ domain is located at the 3′ end of the Gated Primer A. The nucleic acid sequences of the X′ domain, the Stabilization domain(S), the R′ domain, and their complementary sequences, are independent of the Target RNA or DNA sequence; (3) a Primer B, that is a primer nucleic acid strand with sequence P2′; (4) a Primer C, that is a primer nucleic acid strand with sequence P3; (5) an EXPAR template, that contains subsequences forming an EXPAR Trigger′ (X′) domain, a Nicking enzyme recognition site′ (R′) domain, and an EXPAR Trigger′ (X′) domain, respectively, listed in the 5′→3′ direction; (6) a strand-displacing polymerase. For detection of RNA targets, the reaction can use a strand-displacing DNA polymerase with sufficient reverse transcriptase activity (e.g., Bst 2.0 or Bst 3.0) or additionally include (7) a dedicated reverse transcriptase; (8) a nicking enzyme (e.g., Nt.BstNBI) that nicks one strand of a double-stranded DNA at the specific cut site recognized by the nicking enzyme. As mentioned previously, standard nucleic acid reaction components such as reaction buffers, salts, and other additives such as molecular enhancers, excipients, and fluorescent or colorimetric indicators are not depicted in this and the following drawings.

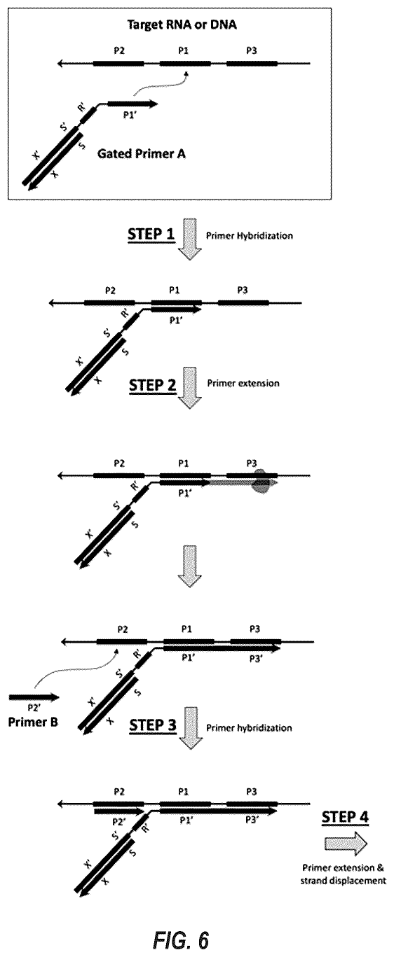

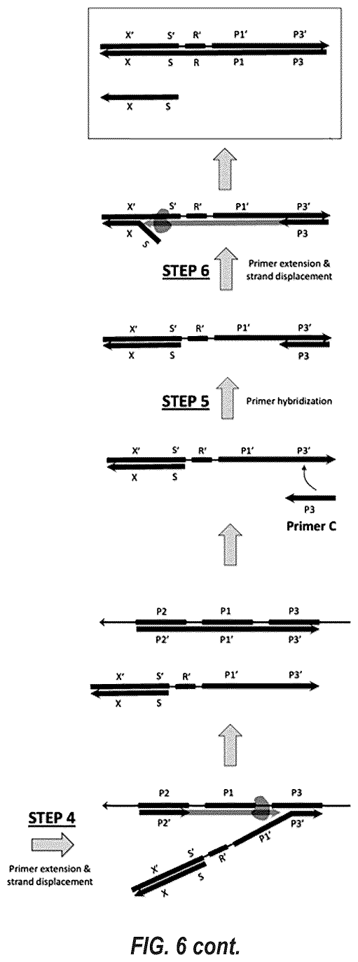

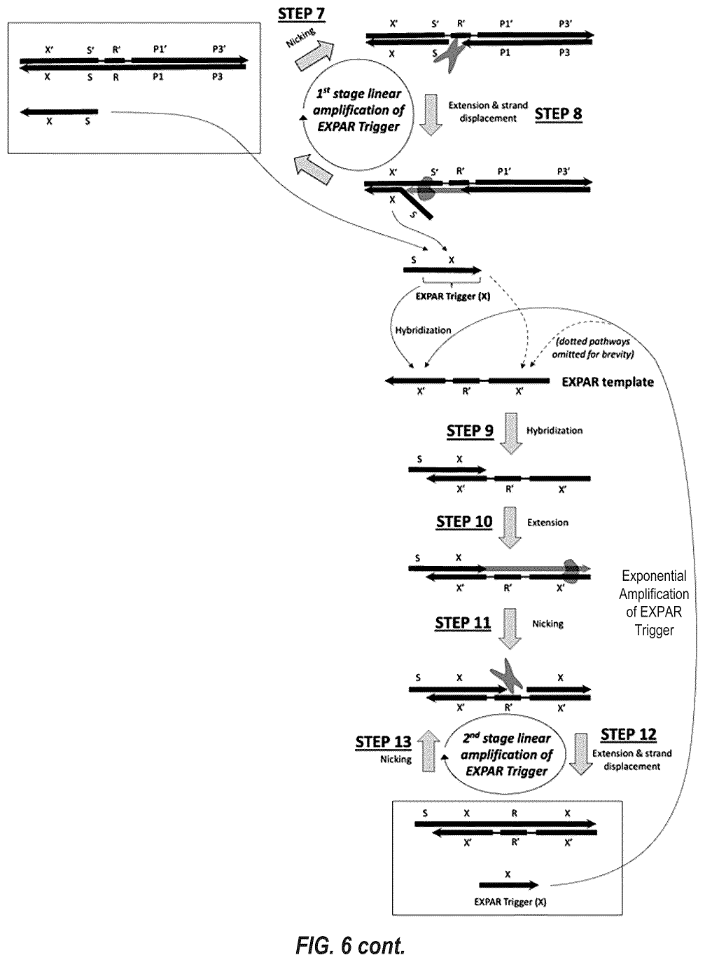

FIG. 6 illustrates the mechanism of an ultra-fast one-pot isothermal nucleic acid amplification reaction based on components listed in FIG. 5. The reaction contains two stages. The first stage is considered as a target transduction stage which converts the detection of a target RNA or DNA strand into linear amplification of a Trigger strand, which is then exponentially amplified in the second stage by an EXPAR reaction. Specifically, in STEP 1, the Gated Primer A hybridizes to the target RNA or DNA strand by binding to the P1 domain. In STEP 2, the 3′ end of the Gated Primer A is extended by polymerase forming a domain P3′ that is complementary to the domain P3 on the target sequence. In STEP 3, the Primer B hybridizes to the target RNA or DNA strand by binding to the P2 domain. In STEP 4, the 3′ end of the Primer B is extended by polymerase, and via primer extension and strand displacement, displacing the extended Gated Primer A (i.e., Gated Primer A with the extended P3′ domain) from the duplex DNA. There may be additional bases attached to the 3′ of the P3′ domain due to further extension by polymerase and for brevity, those bases are not depicted in the drawing. In STEP 5, the Primer C hybridizes to the extended Gated Primer A resulted from STEP 4 by binding to the P3′ domain. In STEP 6, the 3′ end of the Primer C is extended by polymerase forming a double-stranded DNA containing domains P3, P1, R, S, and X in the bottom strand listed in the 5′→3′ direction. In STEP 7, the nicking enzyme recognizes the R′/R domain and nicks the bottom strand. In STEP 8, the polymerase extends the 3′ end of the strand at the nicking site and releases the nucleic acid sequence containing domains S and X (listed in the 5′→3′ direction) by strand displacement and reforms the double-stranded DNA. This duplex DNA serves as a template for repeated nicking, polymerase extension, and displacement reactions, linearly amplifying the nucleic acid sequence containing domains S and X (listed in the 5′→3′ direction) that functions as EXPAR Trigger (X) for next step. In STEP 9, the domain X at the 3′ end of the single-stranded nucleic acid sequence linearly amplified from STEP 8 serves as the EXPAR Trigger (X) and hybridizes to the EXPAR template at the X′ domains. For brevity, the drawing illustrates only the productive reaction pathways corresponding to EXPAR Trigger hybridization at the 3′ end of the EXPAR template because EXPAR Trigger hybridized at the 5′ end of the EXPAR Template does not trigger effective amplification and will be displaced by extension of the EXPAR Trigger hybridized at the 3′ end of the EXPAR Template. In STEP 10, the 3′ end of the EXPAR Trigger X is extended by polymerase forming a double-stranded DNA containing domains X, R, and X in the top strand listed in the 5′→3′ direction. This double-stranded DNA may also contain a single-stranded S domain if the EXPAR Trigger (X) was generated from STEP 8. In STEP 11, the nicking enzyme recognizes the R′/R domain and nicks the top strand. Via repeated nicking, extension, and strand displacement reactions illustrated in STEP 12 and 13, the EXPAR Trigger (X) is linearly amplified. Newly generated EXPAR Trigger (X) can hybridize to more single-stranded EXPAR templates in the reaction, forming duplex DNA complexes that each serves as a linear amplifier of the EXPAR Trigger (X). As a result, this creates a rapid, exponential amplification of the Trigger (X). Note that the newly generated EXPAR Trigger (X) from the STEPS 11-13 do not contain the single-stranded S domain as compared to Trigger sequence generated from STEP 8. However, this does not alter the reaction pathways depicted in STEPS 9-13, and for brevity, is not described separately in the drawing.

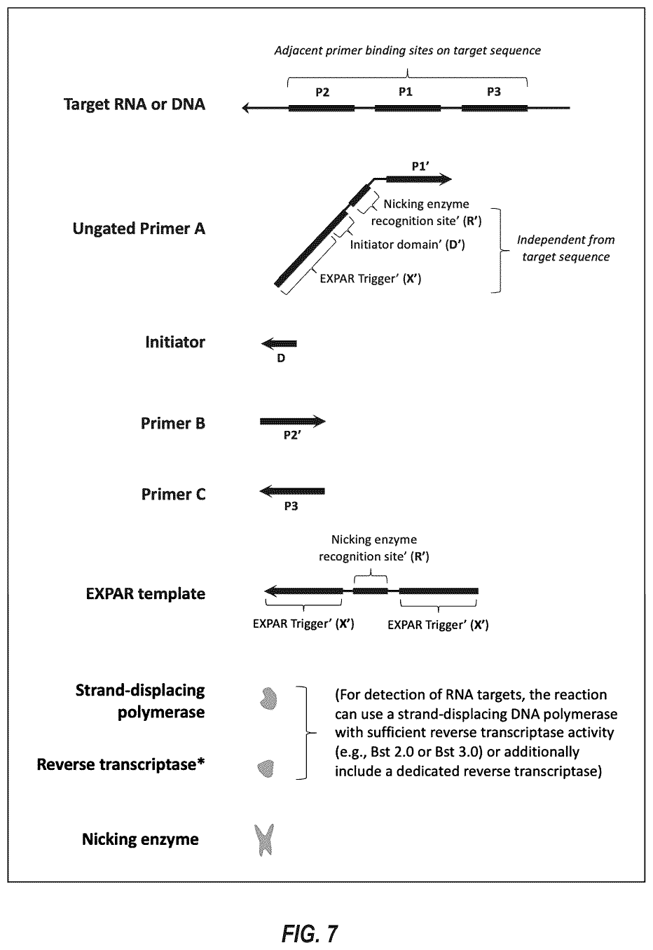

FIG. 7 illustrates the components of another design of an ultra-fast one-pot isothermal nucleic acid amplification reaction. Reaction buffers and salts are not depicted. Compared to the design illustrated in FIG. 5 and FIG. 6, this design features the use of a single-stranded ungated primer which reacts with a single-stranded initiator to form a gated primer required for the downstream reaction. This design eliminates the need to pre-anneal and purify the gated primer. Main components for such a reaction include (1) a Target RNA or DNA sequence, that contains subsequences P3, P1, and P2 (listed in the 5′→3′ direction) as three adjacent primer binding sites; (2) an Ungated Primer A, that is a primer nucleic acid strand containing subsequences forming an EXPAR Trigger′ (X′) domain, an Initiator domain′ (D′), a Nicking enzyme recognition site′ (R′) domain, an EXPAR Trigger′ (X′) domain, a Nicking enzyme recognition site′ (R′) domain, and a P1′ domain, respectively, listed in the 5′→3′ direction. The P1′ domain is located at the 3′ end of the Ungated Primer A. The nucleic acid sequences of the X′, D′, and R′ domains are independent of the Target RNA or DNA sequence; (3) an Initiator, that is a primer nucleic acid strand with sequence D; (4) a Primer B, that is a primer nucleic acid strand with sequence P2′; (5) a Primer C, that is a primer nucleic acid strand with sequence P3; (6) an EXPAR template, that contains subsequences forming an EXPAR Trigger′ (X′) domain, a Nicking enzyme recognition site′ (R′) domain, and an EXPAR Trigger′ (X′) domain, respectively, listed in the 5′→3′ direction; (7) a strand-displacing polymerase. For detection of RNA targets, the reaction can use a strand-displacing DNA polymerase with sufficient reverse transcriptase activity (e.g., Bst 2.0 or Bst 3.0) or additionally include (8) a dedicated reverse transcriptase; (9) a nicking enzyme (e.g., Nt.BstNBI) that nicks one strand of a double-stranded DNA at the specific cut site recognized by the nicking enzyme. As mentioned previously, standard nucleic acid reaction components such as reaction buffers, salts, and other additives such as molecular enhancers, excipients, and fluorescent or colorimetric indicators are not depicted in this and the following drawings.

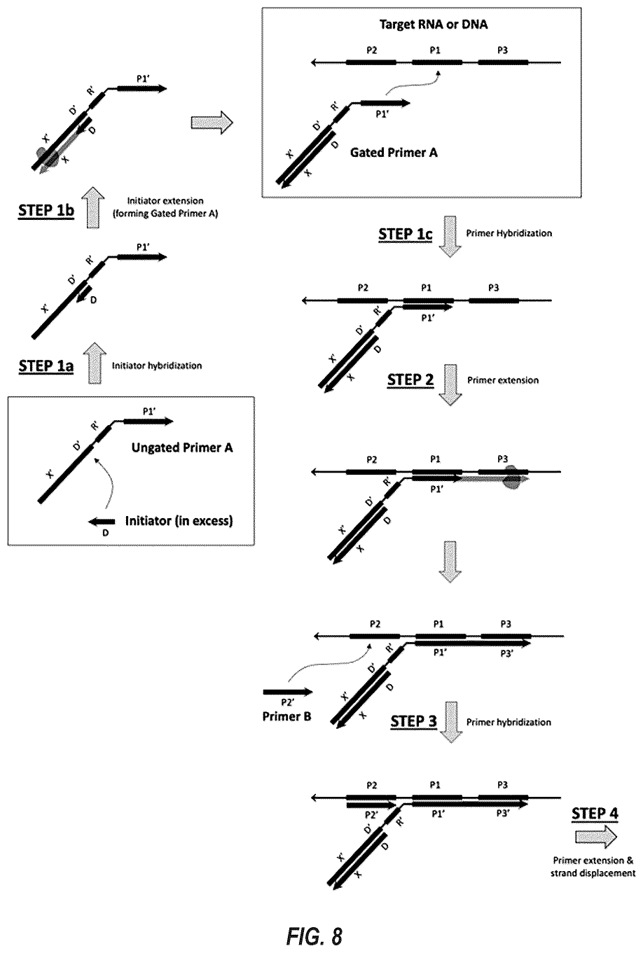

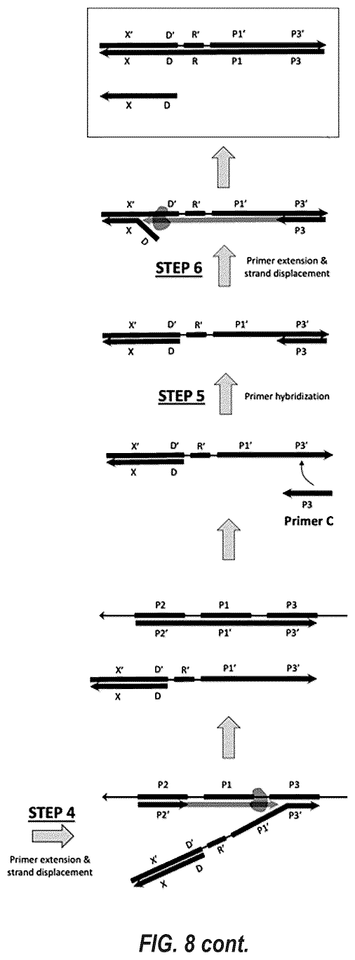

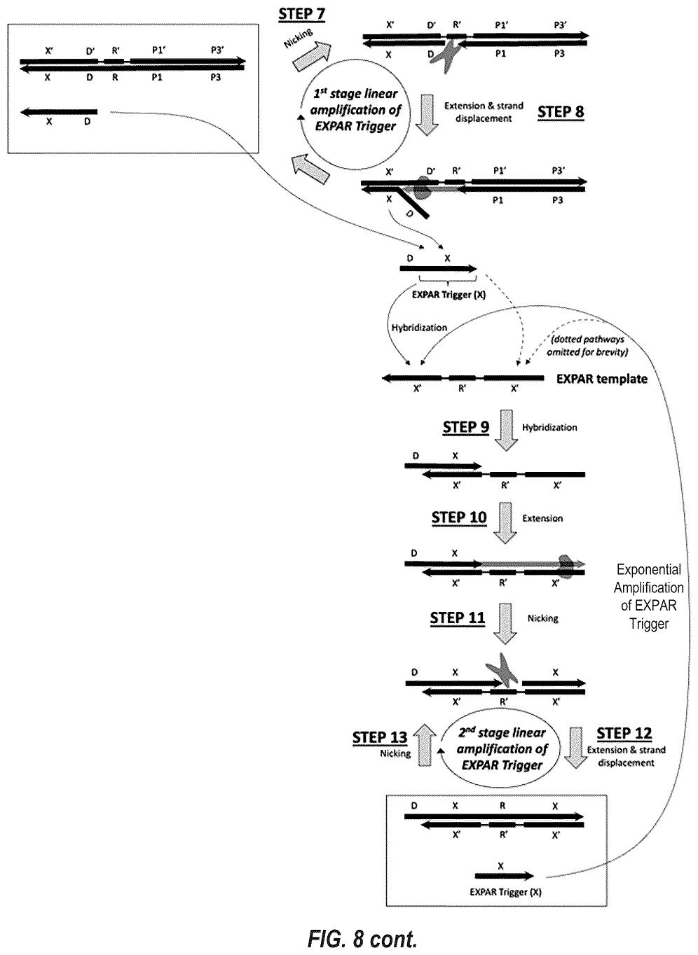

FIG. 8 illustrates the mechanism of an ultra-fast one-pot isothermal nucleic acid amplification reaction based on components listed in FIG. 7. The reaction contains two stages. The first stage is considered as a target transduction stage which converts the detection of a target RNA or DNA strand into linear amplification of a Trigger strand, which is then exponentially amplified in the second stage by an EXPAR reaction. Specifically, in STEP 1a, the Initiator hybridizes to the D′ domain on the Ungated Primer A; In STEP 1b, the 3′ end of the Initiator is extended by polymerase forming a domain X that is complementary to the domain X′ on the Ungated Primer A. This completes the formation of a Gated Primer A. In STEP 1c, the formed Gated Primer A hybridizes to the target RNA or DNA strand by binding to the P1 domain. In STEP 2, the 3′ end of the Gated Primer A is extended by polymerase forming a domain P3′ that is complementary to the domain P3 on the target sequence. In STEP 3, the Primer B hybridizes to the target RNA or DNA strand by binding to the P2 domain. In STEP 4, the 3′ end of the Primer B is extended by polymerase, and via primer extension and strand displacement, displacing the extended Gated Primer A (i.e., Gated Primer A with the extended P3′ domain) from the duplex DNA. There may be additional bases attached to the 3′ of the P3′ domain due to further extension by polymerase and for brevity, those bases are not depicted in the drawing. In STEP 5, the Primer C hybridizes to the extended Gated Primer A resulted from STEP 4 by binding to the P3′ domain. In STEP 6, the 3′ end of the Primer Cis extended by polymerase forming a double-stranded DNA containing domains P3, P1, R, D, and X in the bottom strand listed in the 5′→3′ direction. In STEP 7, the nicking enzyme recognizes the R′/R domain and nicks the bottom strand. In STEP 8, the polymerase extends the 3′ end of the strand at the nicking site and releases the nucleic acid sequence containing domains D and X (listed in the 5′→3′ direction) by strand displacement and reforms the double-stranded DNA. This duplex DNA serves as a template for repeated nicking, polymerase extension, and displacement reactions, linearly amplifying the nucleic acid sequence containing domains D and X (listed in the 5′→3′ direction) that functions as EXPAR Trigger (X) for next step. In STEP 9, the domain X at the 3′ end of the single-stranded nucleic acid sequence linearly amplified from STEP 8 serves as the EXPAR Trigger (X) and hybridizes to the EXPAR template at the X′ domains. For brevity, the drawing illustrates only the productive reaction pathways corresponding to EXPAR Trigger hybridization at the 3′ end of the EXPAR template because EXPAR Trigger hybridized at the 5′ end of the EXPAR Template does not trigger effective amplification and will be displaced by extension of the EXPAR Trigger hybridized at the 3′ end of the EXPAR Template. In STEP 10, the 3′ end of the EXPAR Trigger X is extended by polymerase forming a double-stranded DNA containing domains X, R, and X in the top strand listed in the 5′→3′ direction. This double-stranded DNA may also contain a single-stranded D domain if the EXPAR Trigger (X) was generated from STEP 8. In STEP 11, the nicking enzyme recognizes the R′/R domain and nicks the top strand. Via repeated nicking, extension, and strand displacement reactions illustrated in STEP 12 and 13, the EXPAR Trigger (X) is linearly amplified. Newly generated EXPAR Trigger (X) can hybridize to more single-stranded EXPAR templates in the reaction, forming duplex DNA complexes that each serves as a linear amplifier of the EXPAR Trigger (X). As a result, this creates a rapid, exponential amplification of the Trigger (X). Note that the newly generated EXPAR Trigger (X) from the STEPS 11-13 do not contain the single-stranded D domain as compared to Trigger sequence generated from STEP 8. However, this does not alter the reaction pathways depicted in STEPS 9-13, and for brevity, is not described separately in the drawing.

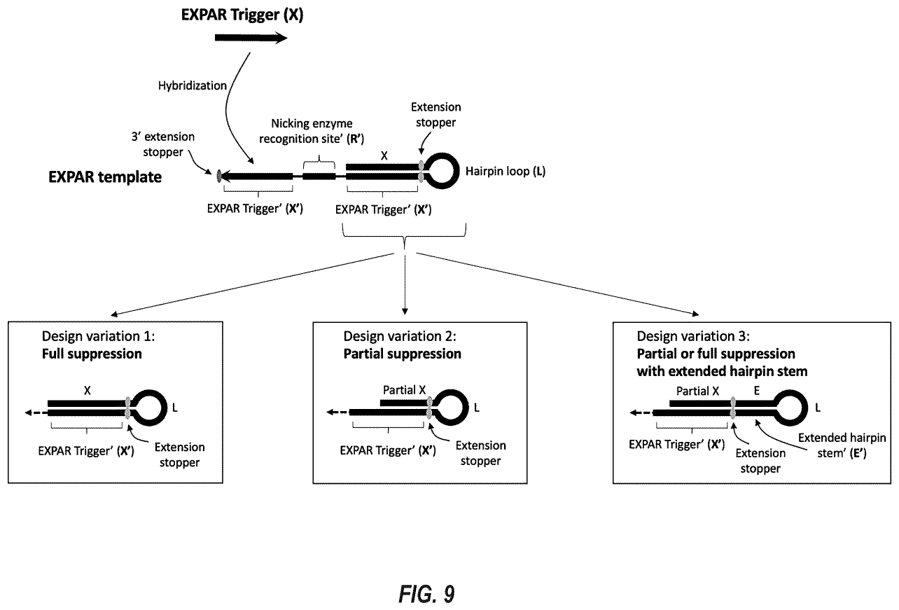

FIG. 9 illustrates example designs of the EXPAR template incorporating mechanisms to improve the amplification efficiency and speed of EXPAR reactions. One of more of these designs can be applied to enhance the different designs of the ultra-fast one-pot isothermal nucleic acid amplification reactions described in the present disclosure. Compared to conventional single-stranded EXPAR template, the present design incorporates a hairpin stem-loop structure at the 5′ end of the template. The stem of the hairpin is designed to sequester the Trigger binding domain at the 5′ end of the EXPAR template, partially or completely. Specifically, this improves amplification efficiency by reducing or eliminating the unproductive binding of Trigger at the 5′ end of the template. In addition, the EXPAR template incorporates a 3′ extension stopper (e.g., 3′ Inverted dT, 3′ Phosphorylation, 3′ Amino Modifier C6 dT, or poly-T tail at the 3′ end) to reduce non-specific amplification. In the example shown, the Trigger X would preferentially hybridize to the X′ domain at the 3′ end of the template, then the polymerase extends the 3′ end of the Trigger X and displaces the domain X or partial X (previously forming the stem of the hairpin structure) before halting at the Extension stopper (e.g., iso-dG-iso-dC base pair). In a design variation, the double-stranded stem of the hairpin structure can be further extended to stabilize the hairpin structure if needed. The nicking enzyme recognizes the R′/R domain and nicks the top strand, generating a single-stranded Trigger X which can spontaneously dissociate from the X′ domain at the 5′ end of the EXPAR template due to the reduced melting temperature after nicking as well as the competitive binding of the X domain (connected to the harpin loop) reforming the double-stranded hairpin stem structure. This forms a linear amplifier of the Trigger X with enhanced efficiency and speed. After dissociation from the template, each newly generated Trigger X can bind to another EXPAR template to generate another enhanced linear amplifier. Together, this enables exponential amplification of the Trigger X with improved efficiency and speed.

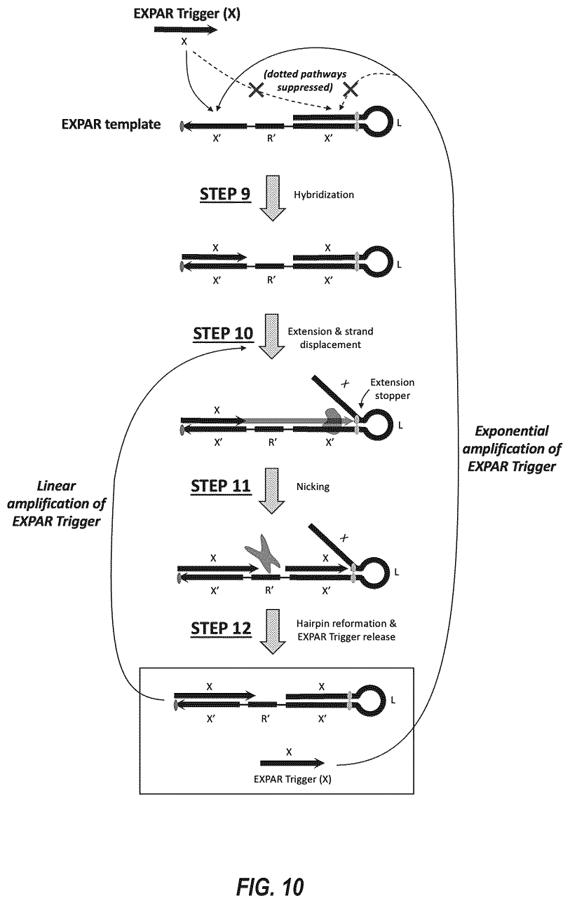

FIG. 10 illustrates the mechanism of the improved isothermal nucleic acid amplification reaction based on one of the enhanced EXPAR template designs illustrated in FIG. 9. In this drawing, the reaction starts at STEP 9 assuming the EXPAR Trigger X has already been generated from upstream reactions, for example, by detection of a target DNA or RNA sequence (STEPS 1-8 from FIG. 2 is one example). In STEP 9, the EXPAR Trigger (X) hybridizes to the EXPAR template preferentially at the X′ domain at the 3′ end of the template owing to the complete or partial sequestration of the X′ domain at the 5′ end of the template as a result of the hairpin structure. This effectively eliminates or reduces unproductive reaction pathways as indicated by the dotted lines. In STEP 10, the 3′ end of the EXPAR Trigger X is extended by polymerase forming a double-stranded DNA containing domains X, R, and X in the top strand listed in the 5′→3′ direction, displacing the domain X or partial X (previously forming the stem of the hairpin structure) before halting at the Extension stopper (e.g., iso-dG-iso-dC base pair). In STEP 11, the nicking enzyme recognizes the R′/R domain and nicks the top strand, generating a single-stranded Trigger X which can spontaneously dissociate from the X′ domain at the 5′ end of the EXPAR template due to the reduced melting temperature after nicking as well as the competitive binding of the X domain (connected to the harpin loop) reforming the double-stranded hairpin stem structure. Via repeated nicking, extension, and strand displacement reactions illustrated in STEPS 10-12, the EXPAR Trigger (X) is linearly amplified. Each newly generated Trigger X can bind to anther EXPAR template to generate another enhanced linear amplifier. As a result, this creates a rapid, exponential amplification of the Trigger (X) with improved efficiency and speed.

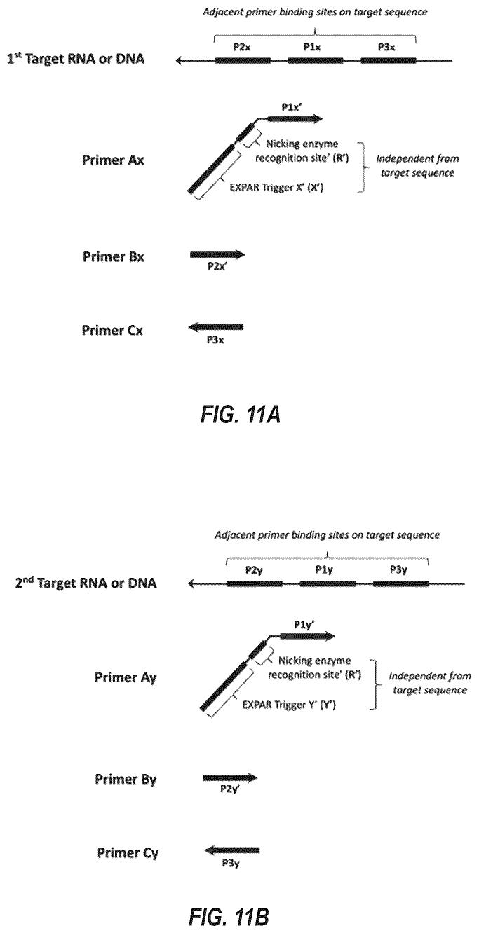

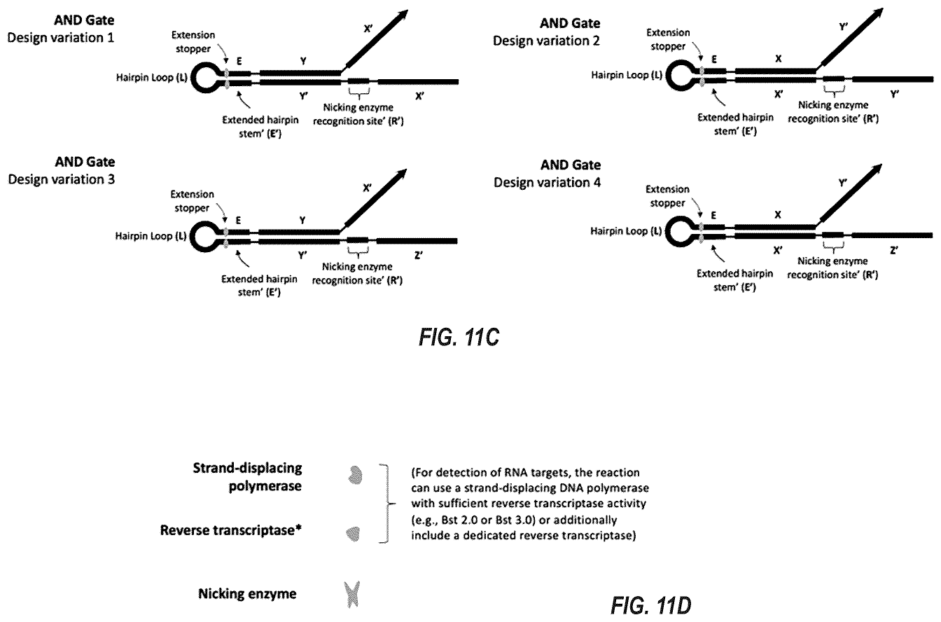

FIGS. 11A-11D illustrates the components of an ultra-fast one-pot isothermal nucleic acid amplification reaction featuring different designs of a molecular logic AND Gate to improve the target detection specificity and multiplexity. Reaction buffers and salts are not depicted. FIG. 11A illustrates reaction components including (1) a 1^{st} Target RNA or DNA sequence, that contains subsequences P3x, P1x, and P2x (listed in the 5′→3′ direction) as three adjacent primer binding sites; (2) a Primer Ax, that is a primer nucleic acid strand containing subsequences forming an EXPAR Trigger X′ (X′) domain, a Nicking enzyme recognition site′ (R′) domain, and a P1x′ domain, respectively, listed in the 5′→3′ direction. The P1x′ domain is located at the 3′ end of the Primer Ax. The nucleic acid sequences of the X′ and R′ domains are independent of the Target RNA or DNA sequence; (3) a Primer Bx, that is a primer nucleic acid strand with sequence P2x′; (4) a Primer Cx, that is a primer nucleic acid strand with sequence P3x. FIG. 11B illustrates reaction components including (5) a 2^{nd} Target RNA or DNA sequence, that contains subsequences P3y, Ply, and P2y (listed in the 5′→3′ direction) as three adjacent primer binding sites; (6) a Primer Ay, that is a primer nucleic acid strand containing subsequences forming an EXPAR Trigger Y′ (Y′) domain, a Nicking enzyme recognition site′ (R′) domain, and a Ply′ domain, respectively, listed in the 5′→3′ direction. The Ply′ domain is located at the 3′ end of the Primer Ay. The nucleic acid sequences of the Y′ and R′ domains are independent of the Target RNA or DNA sequence; (7) a Primer By, that is a primer nucleic acid strand with sequence P2y′; (8) a Primer Cy, that is a primer nucleic acid strand with sequence P3y. FIG. 11C illustrates four different designs of (9) a molecular logic AND Gate, which is pre-annealed and features a hairpin structure.

Specifically, in Design variation 1, the AND Gate is formed by a single-stranded nucleic acid sequence containing domains X′, R′, Y′, E′, L, E, Y, and X′ (listed in the 5′→3′ direction) annealed as a stem-loop hairpin structure via stable hybridization at the E/E′ and Y/Y′ domains. The AND Gate incorporates polymerase Extension stopper (e.g., iso-dG-iso-dC base pair) as indicated near the domain E/E′. In Design variation 2, the AND Gate is formed by a single-stranded nucleic acid sequence containing domains Y′, R′, X′, E′, L, E, X, and Y′ (listed in the 5′→3′ direction) annealed as a stem-loop hairpin structure via stable hybridization at the E/E′ and X/X′ domains. The AND Gate incorporates polymerase Extension stopper (e.g., iso-dG-iso-dC base pair) as indicated near the domain E/E′. In Design variation 3, the AND Gate is formed by a single-stranded nucleic acid sequence containing domains Z′, R′, Y′, E′, L, E, Y, and X′ (listed in the 5′→3′ direction) annealed as a stem-loop hairpin structure via stable hybridization at the E/E′ and Y/Y′ domains. The AND Gate incorporates polymerase Extension stopper (e.g., iso-dG-iso-dC base pair) as indicated near the domain E/E′. In Design variation 4, the AND Gate is formed by a single-stranded nucleic acid sequence containing domains Z′, R′, X′, E′, L, E, X, and Y′ (listed in the 5′→3′ direction) annealed as a stem-loop hairpin structure via stable hybridization at the E/E′ and X/X′ domains. The AND Gate incorporates polymerase Extension stopper (e.g., iso-dG-iso-dC base pair) as indicated near the domain E/E′.

FIG. 11D illustrates (10) a strand-displacing polymerase. For detection of RNA targets, the reaction can use a strand-displacing DNA polymerase with sufficient reverse transcriptase activity (e.g., Bst 2.0 or Bst 3.0) or additionally include (11) a dedicated reverse transcriptase; (12) a nicking enzyme (e.g., Nt.BstNBI) that nicks one strand of a double-stranded DNA at the specific cut site recognized by the nicking enzyme. As mentioned previously, standard nucleic acid reaction components such as reaction buffers, salts, and other additives such as molecular enhancers, excipients, and fluorescent or colorimetric indicators are not depicted in this and the following drawings.

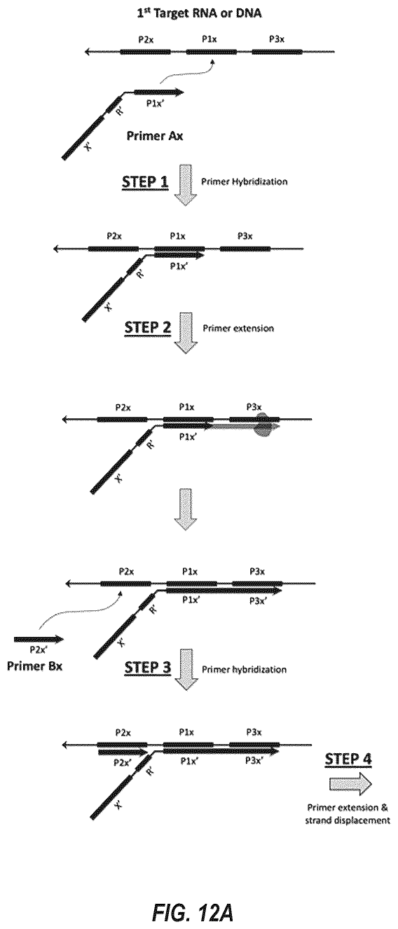

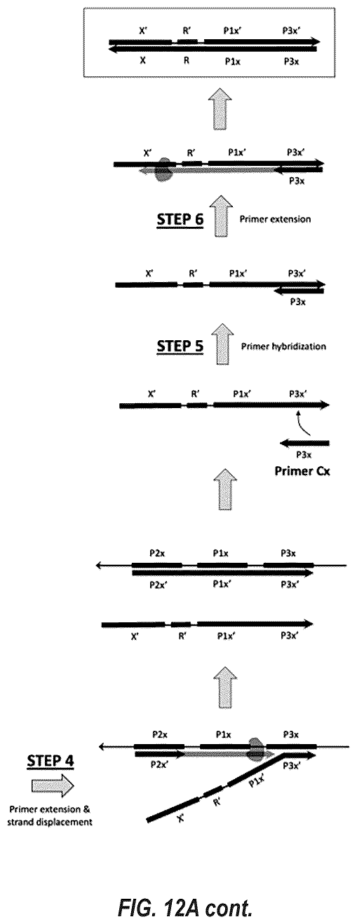

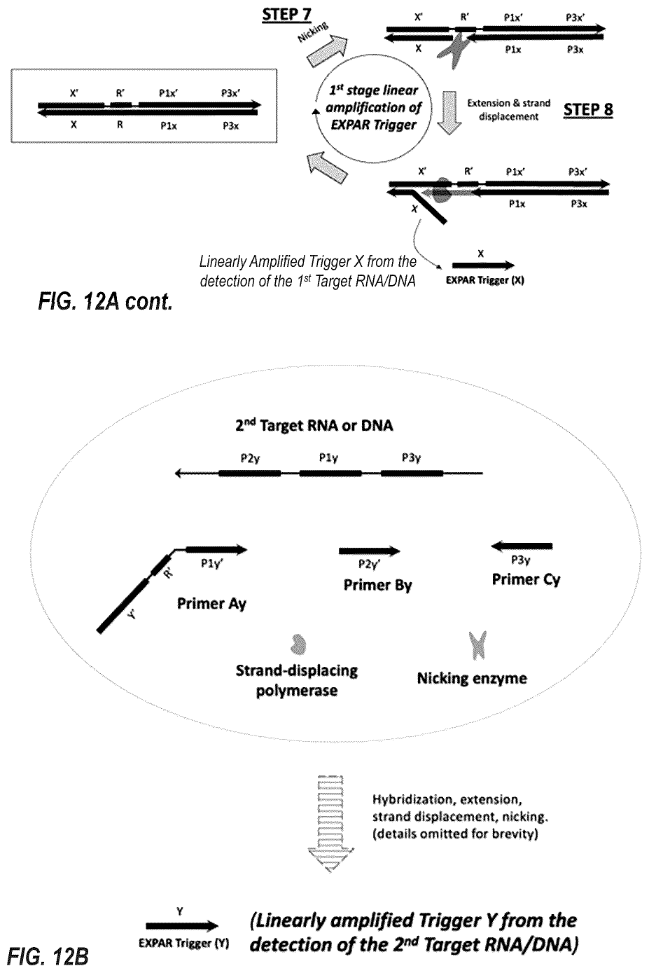

FIGS. 12A and 12B illustrates the mechanism governing the initial phase of an ultra-fast one-pot isothermal nucleic acid amplification reaction based on components listed in FIG. 11. FIG. 12A illustrates the mechanism of generating a linearly amplified EXPAR trigger specific to the detection of the 1^{st} target RNA or DNA. In STEP 1, the Primer Ax hybridizes to the 1^{st} Target RNA or DNA strand by binding to the P1x domain. In STEP 2, the 3′ end of the Primer Ax is extended by polymerase forming a domain P3x′ that is complementary to the domain P3x on the 1^{st} target sequence. In STEP 3, the Primer Bx hybridizes to the 1^{st} Target RNA or DNA strand by binding to the P2x domain. In STEP 4, the 3′ end of the Primer Bx is extended by polymerase, and via primer extension and strand displacement, displacing the extended Primer Ax (i.e., Primer Ax with the extended P3x′ domain) from the duplex DNA. This results in a single-stranded sequence that contains domains X′, R′, P1x′, and P3x′ listed in the 5′→3′ direction. There may be additional bases attached to the 3′ end of the P3x′ domain due to further extension by polymerase and for brevity, those bases are not depicted in the drawing. In STEP 5, the Primer Cx hybridizes to the single-stranded DNA generated from STEP 4 by binding to the P3x′ domain. In STEP 6, the 3′ end of the Primer Cx is extended by polymerase forming a double-stranded DNA containing domains P3x, P1x, R, and X in the bottom strand listed in the 5′→3′ direction. In STEP 7, the nicking enzyme recognizes the R′/R domain and nicks the bottom strand. In STEP 8, the polymerase extends the 3′ end of the strand at the nicking site and releases the nucleic acid sequence X by strand displacement and reforms the double-stranded DNA. This duplex DNA serves as a template for repeated nicking, polymerase extension, and displacement reactions, linearly amplifying the nucleic acid sequence X that functions as EXPAR Trigger (X) for next step. FIG. 12B illustrates the mechanism of generating a linearly amplified EXPAR trigger specific to the detection of the 2^{nd} target RNA or DNA. The reaction mechanism is similar to the mechanism illustrated in FIG. 12A and is omitted for brevity.

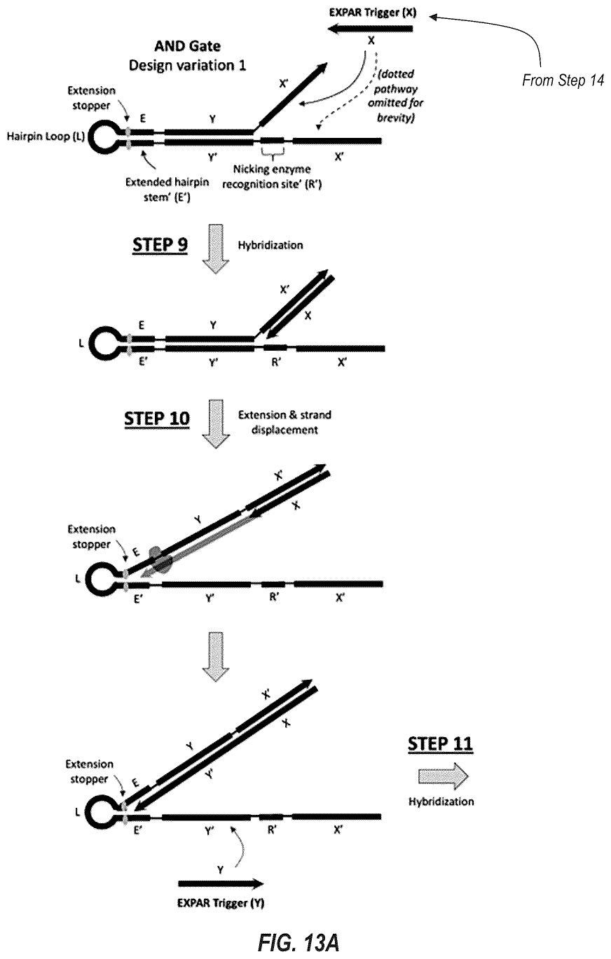

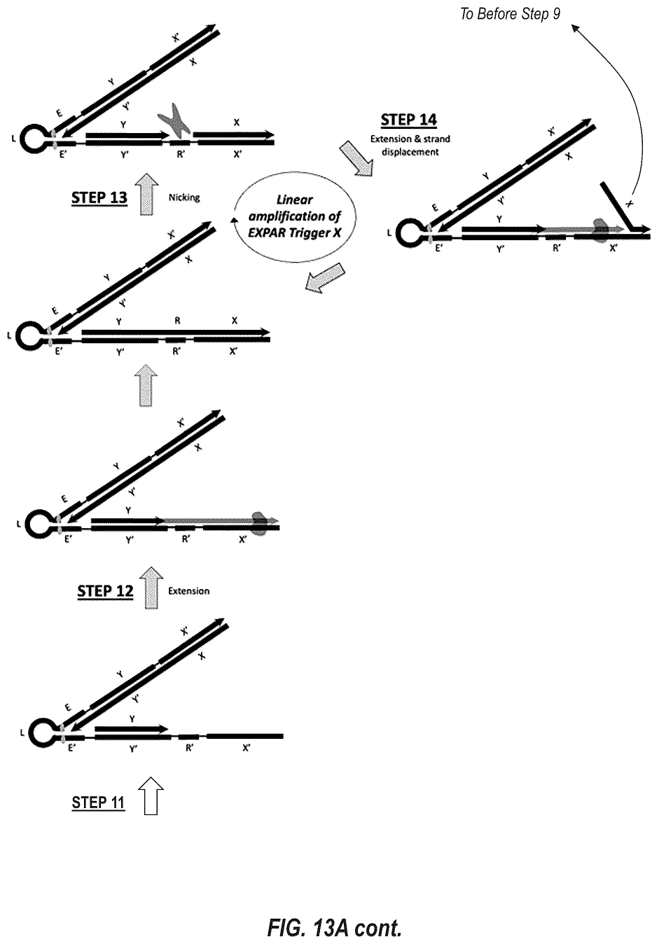

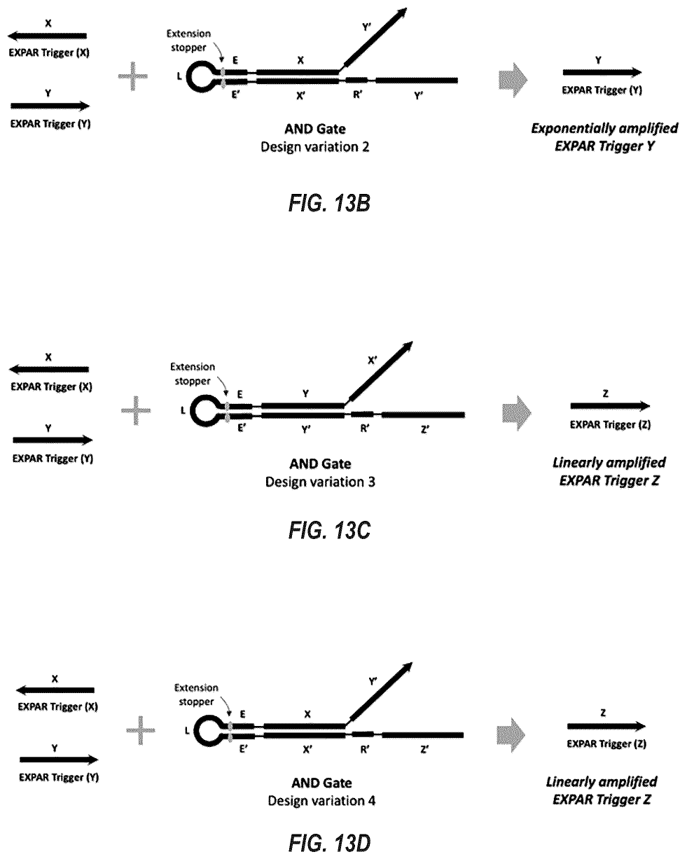

FIGS. 13A-13D illustrate the mechanism governing the second phase of an ultra-fast one-pot isothermal nucleic acid amplification reaction based on components listed in FIG. 11. Reactions illustrated in FIG. 13 continue from the linear amplification of both EXPAR Trigger X and Trigger Y illustrated in FIG. 12. Specifically, FIG. 13A illustrates the mechanism of exponentially amplifying the EXPAR Trigger X based on the AND Gate Design variation 1 illustrated in FIG. 11. This exponential amplification reaction requires the presence of both EXPAR Trigger (X) and EXPAR Trigger (Y) generated from the successful detection of both targets (i.e., the 1^{st} and 2^{nd} Target RNA or DNA illustrated in FIG. 12). In STEP 9, The EXPAR Trigger (X) hybridizes to the X′ domains on the AND Gate (unproductive reaction pathway is omitted for brevity). In STEP 10, the 3′ end of the EXPAR Trigger (X) hybridized to the X′ domain at the 3′ end of the AND Gate is extended by polymerase and displaces the originally bound domains Y′ and E′ (previously forming the stem of the hairpin structure) of the AND Gate before halting at the Extension stopper (e.g., iso-dG-iso-dC base pair). This process exposes the single-stranded domain Y′ on the AND Gate for available binding to the EXPAR Trigger (Y) in the next step. In STEP 11, the EXPAR Trigger (Y) hybridizes to the newly exposed Y′ domain on the AND Gate. In STEP 12, the 3′ end of the EXPAR Trigger (Y) is extended by polymerase forming a double-stranded DNA containing domains Y, R, and X in the top strand listed in the 5′→3′ direction. In STEP 13, the nicking enzyme recognizes the R′/R domain and nicks the top strand. Via repeated nicking, extension, and strand displacement reactions illustrated in STEP 13 and 14, the EXPAR Trigger (X) is linearly amplified. Newly generated EXPAR Trigger (X) can hybridize to more AND Gates (those with the X′ domain at the 3′ end freely available) in the reaction, forming additional DNA complexes that each serves as a linear amplifier of the EXPAR Trigger (X). As a result, this creates a rapid, exponential amplification of the Trigger (X).

FIG. 13B illustrates the mechanism of exponentially amplifying the EXPAR Trigger Y based on the AND Gate Design variation 2 illustrated in FIG. 11 following the linear amplification of both EXPAR Trigger X and Trigger Y from FIG. 12. FIGS. 13C and 13D illustrate the mechanisms of linearly amplifying an EXPAR Trigger Z based on the AND Gate Design variation 3 and 4, respectively, illustrated in FIG. 11 following the linear amplification of both EXPAR Trigger X and Trigger Y from FIG. 12. Detailed reaction pathways for FIG. 13B-D are omitted for brevity.

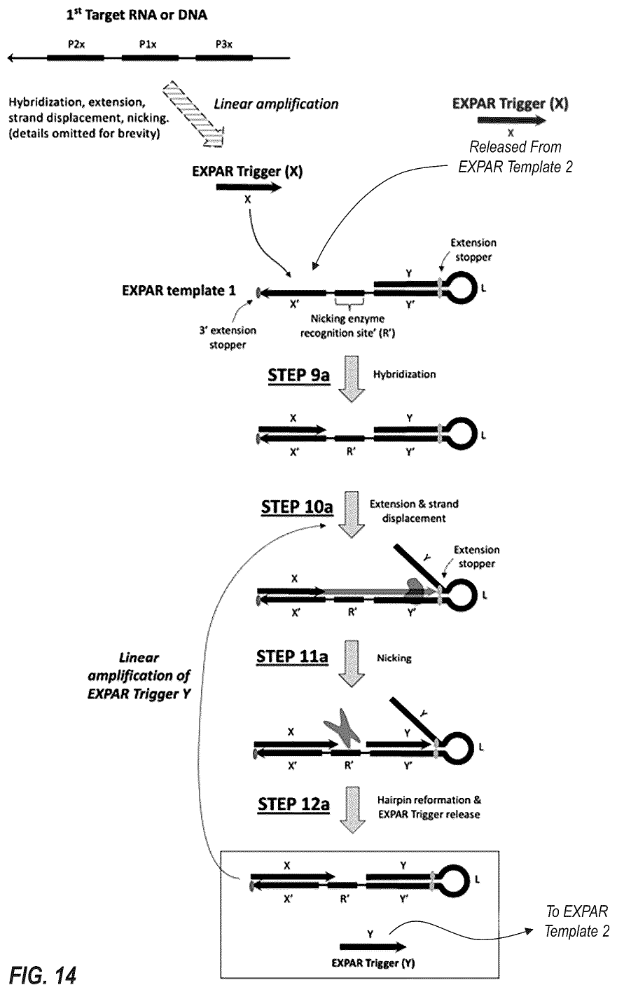

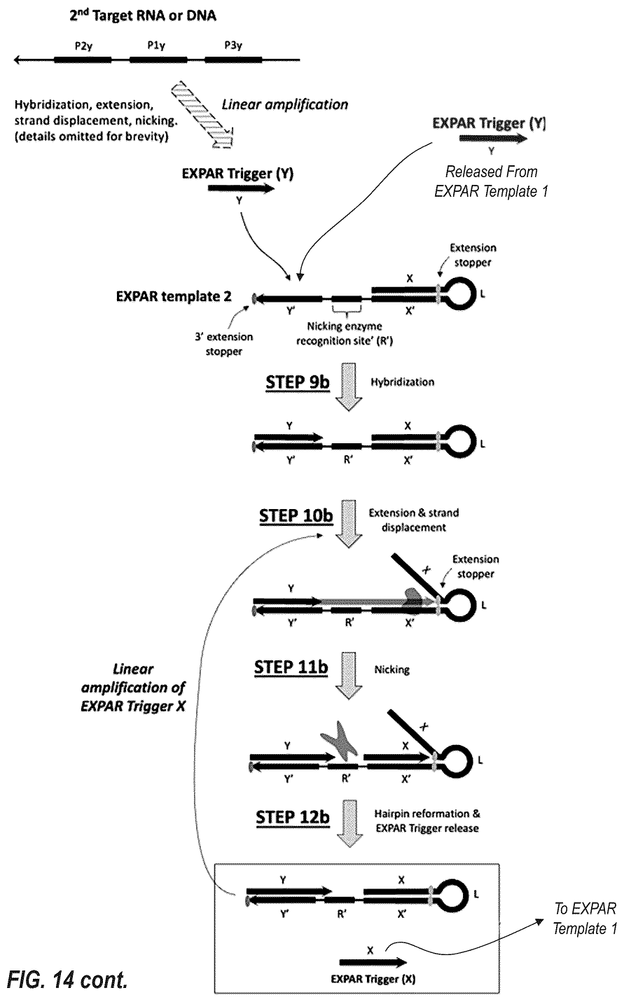

FIG. 14 illustrates the design and mechanism of exponential cross-amplification reaction initiated by the presence of one or both types of the EXPAR triggers linearly amplified from the specific detection of the 1^{st} and the 2^{nd} RNA or DNA targets, respectively. This reaction essentially computes a logic OR between detection of the 1^{st} and the 2^{nd} RNA or DNA target. This design eliminates the use of duplicate domains on the EXPAR templates, offering advantages in mitigating the common occurrence of non-specific amplifications in EXPAR reactions. In STEP 9a, the EXPAR Trigger (X) generated from the detection of the 1^{st} Target RNA or DNA hybridizes to the X′ domain on the EXPAR template 1. Note that the EXPAR templates shown in this drawing are pre-annealed and features a hairpin structure previously described in FIG. 9. In STEP 10a, the 3′ end of the EXPAR Trigger (X) is extended by polymerase and displaces the originally bound domain Y (previously forming the stem of the hairpin structure) of the EXPAR template 1 before halting at the Extension stopper (e.g., iso-dG-iso-dC base pair). In STEP 11a, the nicking enzyme recognizes the R′/R domain and nicks the top strand, generating a single-stranded EXPAR Trigger (Y) which can spontaneously dissociate from the Y′ domain of the EXPAR template 1 due to the reduced melting temperature after nicking as well as the competitive binding of the Y domain (connected to the harpin loop) reforming the double-stranded hairpin stem structure (STEP 12a). This DNA complex serves as a template for repeated nicking, polymerase extension, and displacement reactions (STEPS 10a-12a), linearly amplifying the nucleic acid sequence Y that functions as EXPAR Trigger (Y) for the next step. In STEP 9b, the EXPAR Trigger (Y) generated from the detection of the 2^{nd} Target RNA or DNA as well as the EXPAR Trigger (Y) generated from STEP 12a can both hybridize to the Y′ domain on the EXPAR template 2. In STEP 10b, the 3′ end of the EXPAR Trigger (Y) is extended by polymerase and displaces the originally bound domain X (previously forming the stem of the hairpin structure) of the EXPAR template 2 before halting at the Extension stopper (e.g., iso-dG-iso-dC base pair). In STEP 11b, the nicking enzyme recognizes the R′/R domain and nicks the top strand, generating a single-stranded EXPAR Trigger (X) which can spontaneously dissociate from the X′ domain of the EXPAR template 2 due to the reduced melting temperature after nicking as well as the competitive binding of the X domain (connected to the harpin loop) reforming the double-stranded hairpin stem structure (STEP 12b). This DNA complex serves as a template for repeated nicking, polymerase extension, and displacement reactions (STEPS 10b-12b), linearly amplifying the nucleic acid sequence X that functions as EXPAR Trigger (X) to react with more EXPAR template 1 (those with the X′ domain at the 3′ end freely available) in the reaction.

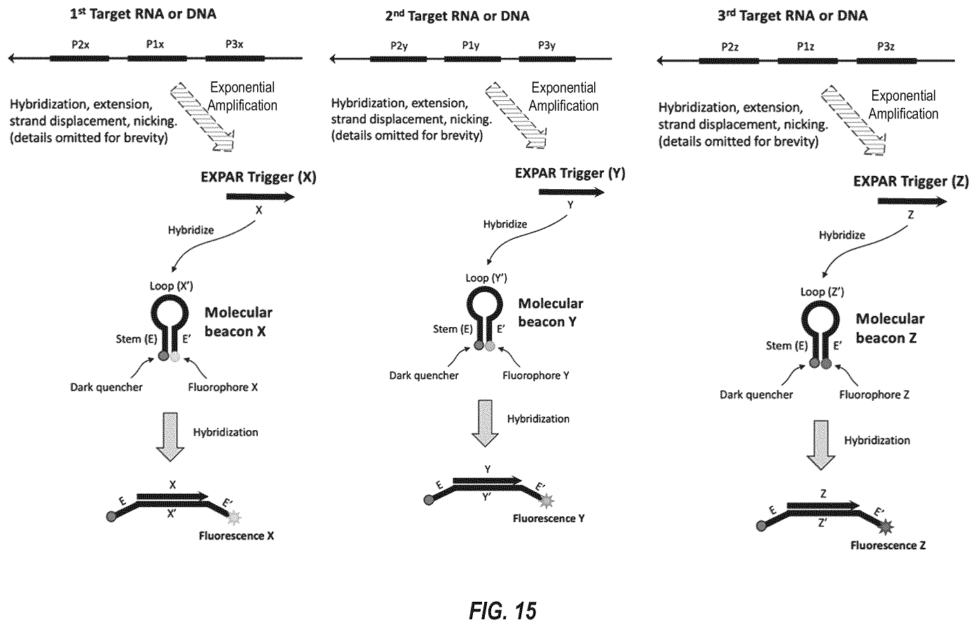

FIG. 15 illustrates example designs and mechanisms of multiplexed detection of different targets based on an ultra-fast one-pot isothermal nucleic acid amplification reaction combined with molecular beacons that react specifically to different EXPAR triggers amplified from the detection of different RNA and/or DNA targets. For example, EXPAR Trigger (X) is generated and exponentially amplified upon detection of the 1^{st} Target RNA or DNA. The resulting EXPAR Trigger X hybridizes to the Loop (X′) domain of the Molecular beacon X leading to dehybridization of the Stem domain. This results in sufficient separation of the quencher and the fluorophore X on the molecular beacon, generating fluorescence specific to the spectrum of fluorophore X. Similarly, successful detection of the 2^{nd} Target RNA or DNA results in the generation and exponential amplification of the EXPAR Trigger (Y), which hybridizes to the Loop (Y′) domain of the Molecular beacon Y leading to dehybridization of the Stem domain. This results in sufficient separation of the quencher and the fluorophore Y on the molecular beacon, generating fluorescence specific to the spectrum of fluorophore Y. Also similarly, successful detection of the 3^{rd} Target RNA or DNA results in the generation and exponential amplification of the EXPAR Trigger (Z), which hybridizes to the Loop (Z′) domain of the Molecular beacon Z leading to dehybridization of the Stem domain. This results in sufficient separation of the quencher and the fluorophore Z on the molecular beacon, generating fluorescence specific to the spectrum of fluorophore Z.

The same concept can be extended to enable specific detection of additional targets. Multiplexed readout can be achieved by fluorescence-generating mechanisms other than molecular beacons. Examples include but are not limited to Scorpions probes, and strand-displacement probes such as assimilating probes, detection of amplification by releasing of quenching (DARQ) probes, oligonucleotide strand exchange (OSD) probes, etc. Note that the designs described herein are illustrative examples and are not intended to be limiting or exclusive. For example, the 1^{st} and 2^{nd} Target RNA or DNA can be nucleic acid sequences from different regions on the same target genomic sequence, and the molecular beacon X and the molecular beacon Y can be designed with the same type of quencher/fluorophore pair. Upon detection of the 1^{st} and 2^{nd} Target RNA or DNA, the generated EXPAR Trigger (X) and Trigger (Y) hybridizes to the molecular beacon X and molecular beacon Y, respectively, both generating the same type of fluorescence thereby enhancing the detection sensitivity. In another example, detection of the 1^{st} and 2^{nd} Target RNA or DNA can lead to the generation and exponential amplification of the same type of Trigger that subsequently react with only one type of fluorescence reporter.

### Example Aspects

The following list of clauses represents a non-exhaustive list of example features according to the disclosed compositions and methods:

Clause 1. A composition formulated to enable detection of a target nucleic acid sequence using isothermal amplification, the composition comprising: a primer A comprising, in the 5′ to 3′ direction, an X′ domain, an R′ domain that is reverse complementary to a nicking enzyme recognition site R, and a P1′ domain that is reverse complementary to a P1 domain of the target sequence, wherein the X′ and R′ domains are independent from the target sequence; a primer B comprising a P2′ domain that is reverse complementary to a P2 domain of the target sequence, wherein P2 is located 3′ of P1 in the target sequence; a primer C comprising a P3 domain that is equivalent to a P3 domain of the target sequence, wherein P3 is located 5′ of P1 in the target sequence; a polymerase enzyme that exhibits strand-displacing functionality; a nicking enzyme that recognizes a double-stranded site of hybridized R and R′ domains and nicks the R domain; and an EXPAR template, wherein primers A, B, and C, when exposed to the P1, P2, and P3 domains of the target sequence, trigger a series of reactions that result in a double-stranded fragment used to carry out a first amplification stage that linearly amplifies the EXPAR trigger X via repeated cycles of nicking, strand extension, and strand displacement, and wherein the EXPAR template is configured to hybridize with the EXPAR trigger X to initiate a second amplification stage that exponentially amplifies the EXPAR trigger X via repeated cycles of nicking, strand extension, and strand displacement.

Clause 2. The composition of clause 1, wherein: primers A, B, and C, when exposed to the P1, P2, and P3 domains of the target sequence, trigger a series of reactions that enable the polymerase to form the double-stranded fragment, wherein a first strand of the double-stranded fragment comprises, in the 5′ to 3′ direction, domains P3, P1, R, and X; the nicking enzyme, when exposed to the double-stranded fragment, nicks within the R domain of the first strand to form a new 3′ end upstream of the X domain; the polymerase, when exposed to the new 3′ end, (i) extends the new 3′ end, (ii) displaces the X domain to form the single-stranded EXPAR trigger X, and (iii) reforms the double-stranded fragment; and repeated nicking, strand extension, and strand displacement at the double-stranded fragment define the first amplification stage that linearly amplifies the EXPAR trigger X.

Clause 3. The composition of any preceding clause, wherein the EXPAR template comprises, in the 3′ to 5′ direction, a first (3′ most) X′ domain, an R′ domain, and a second (5′ most) X′ domain.

Clause 4. The composition of clause 1, wherein: excess primer A functions as EXPAR template, and further comprises an additional R′ domain located 5′ of the X′ domain and an additional X′ domain located 5′ of the additional R′ domain; following the series of reactions that enable formation of the double-stranded fragment, a first strand of the double-stranded fragment comprises, in the 5′ to 3′ direction, domains P3, P1, 5′ most R, 5′ most X, 3′ most R, and 3′ most X; the nicking enzyme, when exposed to the double-stranded fragment, nicks within the 5′ most R domain and nicks within the 3′ most R domain of the first strand to form two new 3′ ends; the polymerase, when exposed to the new 3′ ends, (i) extends the new 3′ ends, (ii) displaces the 3′ most X domain and the 5′ most X domain to respectively form the single-stranded EXPAR trigger X and an EXPAR trigger X analog, and (iii) reforms the double-stranded fragment, wherein the EXPAR trigger X analog comprise an X domain and possibly an additional or partial X domain; and the EXPAR template is configured to hybridize with the EXPAR trigger X and/or EXPAR trigger X analog to initiate the second amplification stage that exponentially amplifies the EXPAR trigger X, via repeated cycles of nicking, strand extension, and strand displacement.

Clause 5. The composition of any preceding clause, wherein the polymerase enzyme exhibits reverse transcriptase functionality, such as wherein the polymerase comprises Bst 2.0 or Bst 3.0.

Clause 6. The composition of clause 5, wherein the composition omits reverse transcriptase enzyme.

Clause 7. The composition of any of clauses 1-5, further comprising a reverse transcriptase enzyme.

Clause 8. The composition of any preceding clause, wherein the composition is formulated to enable a one-pot reaction.

Clause 9. The composition of clause 8, wherein one or more components of the composition are in solution.

Clause 10. The composition of clause 8 or clause 9, wherein one or more components of the composition are in an air-dried or freeze-dried format.

Clause 11. The composition of any preceding clause, wherein: the primer A further comprises an S′ domain, located 5′ of the R′ domain and 3′ of the X′ domain, that is reverse complementary to a stabilization domain S, and further comprises a strand that comprises S and X domains and is hybridized to the S′ and X′ domains to form a gated primer; following the series of reactions that enable formation of the double-stranded fragment, a first strand of the double-stranded fragment comprises, in the 5′ to 3′ direction, domains P3, P1, R, S, and X, the nicking enzyme, when exposed to the double-stranded fragment, nicks within the R domain of the first strand to form a new 3′ end upstream of the S and X domains, the polymerase, when exposed to the new 3′ end, (i) extends the new 3′ end, (ii) displaces the S and X domains to form a single-stranded EXPAR trigger X with 5'S domain, and (iii) reforms the double-stranded fragment, and the EXPAR template is configured to hybridize with the EXPAR trigger X, with or without 5'S domain, to initiate the second amplification stage that exponentially amplifies the EXPAR trigger X, without the S domain, via repeated cycles of nicking, strand extension, and strand displacement.

Clause 12. The composition of any preceding clause, further comprising an initiator D, wherein: the primer A further comprises a D′ domain, located 5′ of the R′ domain and 3′ of the X′ domain, that is reverse complementary to the initiator D; the polymerase, when initiator D is hybridized to domain D′ of primer A, extends the initiator D to form a strand that comprises D and X domains hybridized to the D′ and X′ domains of the primer A to form a gated primer; following the series of reactions that enable formation of the double-stranded fragment, a first strand of the double-stranded fragment comprises, in the 5′ to 3′ direction, domains P3, P1, R, D, and X, the nicking enzyme, when exposed to the double-stranded fragment, nicks within the R domain of the first strand to form a new 3′ end upstream of the D and X domains, the polymerase, when exposed to the new 3′ end, (i) extends the new 3′ end, (ii) displaces the D and X domains to form a single-stranded EXPAR trigger X with 5′ D domain, and (iii) reforms the double-stranded fragment, and the EXPAR template is configured to hybridize with the EXPAR trigger X, with or without 5′ D domain, to initiate the second amplification stage that exponentially amplifies the EXPAR trigger X, without the D domain, via repeated cycles of nicking, strand extension, and strand displacement.

Clause 13. The composition of any preceding clause, wherein the EXPAR template is configured to form a hairpin loop that limits hybridization of the EXPAR trigger X to the 5′ most X′ domain of the EXPAR template.

Clause 14. The composition of clause 13, wherein the EXPAR template comprises a loop-forming domain L located 5′ of the 5′ most X′ domain and an X or partial X domain located 5′ of the loop-forming domain to enable formation of the hairpin loop.

Clause 15. The composition of clause 14, wherein the EXPAR template further comprises an extended hairpin stem domain E located 5′ of the L domain and an E′ domain that is reverse complementary to the E domain and is located 3′ of the L domain, the E and E′ domains forming an extended hairpin stem.

Clause 16. The composition of any of clauses 14-15, wherein the EXPAR template further comprises an extension stopper to prevent the polymerase from driving extension into the loop-forming domain L when the EXPAR trigger X hybridizes to the 3′ most X′ domain of the EXPAR template and is extended therefrom.

Clause 17. The composition of clause 16, wherein the extension stopper comprises an extension stopping base pair such as an iso-dG-iso-dC base pair, wherein one nucleotide of the base pair is located between the X domain and the L domain of the EXPAR template, and one nucleotide of the base pair is located between the 5′ most X′ domain and the L domain of the EXPAR template.

Clause 18. The composition of any preceding clause, wherein the EXPAR template comprises an extension stopper at its 3′ end.

Clause 19. The composition of clause 18, wherein the extension stopper comprises a 3′ Inverted dT, 3′ Phosphorylation, 3′ Amino Modifier C6 dT, poly-T tail, or other 3′ modifications that block polymerase extension.

Clause 20. The composition of any preceding clause, further comprising a labeled probe configured to generate a fluorescence signal in response to interaction with the EXPAR trigger X.

Clause 21. The composition of clause 20, wherein the labelled probe comprises an X′ domain for hybridization with the EXPAR trigger X.

Clause 22. The composition of clause 21, wherein the labeled probe comprises a quencher and a fluorophore that are moved apart from one another upon hybridization of the EXPAR trigger X to the X′ domain of the probe.

Clause 23. The composition of any of clauses 20-22, wherein the labeled probe is a molecular beacon, scorpion probe, strand-displacement probe such as an assimilating probe, detection of amplification by releasing of quenching (DARQ) probe, or oligonucleotide strand exchange (OSD) probe, or other labeled probe capable of generating fluorescence in response to exposure to the EXPAR trigger X.

Clause 24. A multiplex composition formulated to enable detection of two or more target nucleic acid sequences using isothermal amplification, the composition comprising: a first composition as in any preceding clause, formulated to enable detection of a first target sequence by amplifying a first EXPAR trigger as a result of detection of the first target sequence; a second composition as in any preceding claim, formulated to enable detection of a second target sequence by amplifying a second EXPAR trigger as a result of detection of the second target sequence; and optionally, one or more additional compositions as in any preceding claim each respectively formulated to enable detection of a respective target sequence by amplifying a respective EXPAR trigger as a result of detection of the respective target sequence.

Clause 25. The multiplex composition of clause 24, wherein the first EXPAR trigger and the second EXPAR trigger are the same.

Clause 26. The multiplex composition of clause 24, wherein the first EXPAR trigger and the second EXPAR trigger are different.

Clause 27. The multiplex composition of clause 26, the multiplex composition comprising a first labeled probe configured to generate a first fluorescence signal in response to interaction with the first EXPAR trigger, and a second labeled probe configured to generate a second, different fluorescence signal in response to interaction with the second EXPAR trigger.

Clause 28. The multiplex composition of clause 26, the multiplex composition comprising a first labeled probe configured to generate a fluorescence signal in response to interaction with the first EXPAR trigger, and a second labeled probe configured to generate the same fluorescence signal in response to interaction with the second EXPAR trigger.

Clause 29. A composition formulated to enable detection of first and second target nucleic acid sequences through isothermal amplification only when both the first and second target sequences are present within a sample, the composition comprising: (1) a first primer set comprising a primer Ax comprising, in the 5′ to 3′ direction, an X′ domain that is reverse complementary to a first EXPAR trigger X, an R′ domain that is reverse complementary to a nicking enzyme recognition site R, and a P1x′ domain that is reverse complementary to a P1x domain of the first target sequence, wherein the X′ and R′ domains are independent from the first target sequence; a primer Bx comprising a P2x′ domain that is reverse complementary to a P2x domain of the first target sequence, wherein P2x is located 3′ of P1x in the first target sequence; a primer Cx comprising a P3x domain that is equivalent to a P3x domain of the first target sequence, wherein P3x is located 5′ of P1x in the first target sequence, wherein the primers Ax, Bx, and Cx, when exposed to the P1x, P2x, and P3x domains of the target sequence, trigger a series of reactions that result in a first double-stranded fragment used to linearly amplify the EXPAR trigger X via repeated cycles of nicking, strand extension, and strand displacement; (2) a second primer set comprising a primer Ay comprising, in the 5′ to 3′ direction, a Y′ domain that is reverse complementary to a second EXPAR trigger Y, an R′ domain that is reverse complementary to a nicking enzyme recognition site R, and a Ply′ domain that is reverse complementary to a Ply domain of the second target sequence, wherein the Y′ and R′ domains are independent from the second target sequence; a primer By comprising a P2y′ domain that is reverse complementary to a P2y domain of the second target sequence, wherein P2y is located 3′ of Ply in the second target sequence; a primer Cy comprising a P3y domain that is equivalent to a P3y domain of the second target sequence, wherein P3y is located 5′ of Ply in the second target sequence; wherein the primers Ay, By, and Cy, when exposed to the Ply, P2y, and P3y domains of the target sequence, trigger a series of reactions that result in a second double-stranded fragment used to linearly amplify the EXPAR trigger Y via repeated cycles of nicking, strand extension, and strand displacement; (3) a polymerase enzyme that exhibits strand-displacing functionality; (4) a nicking enzyme that recognizes a double-stranded site of hybridized R and R′ domains and nicks the R domain; and (5) an EXPAR template configured as an AND logic gate for generating additional EXPAR trigger sequences via subsequent linear or exponential amplification upon exposure to both the EXPAR trigger X and the EXPAR trigger Y.

Clause 30. The composition of clause 29, wherein: primers Ax, Bx, and Cx, when exposed to the P1x, P2x, and P3x domains of the first target sequence, trigger a series of reactions that enable the polymerase to form the first double-stranded fragment, wherein a strand of the first double-stranded fragment comprises, in the 5′ to 3′ direction, domains P3x, P1x, R, and X; the nicking enzyme, when exposed to the first double-stranded fragment, nicks within the R domain to form a new 3′ end upstream of the X domain; the polymerase, when exposed to the new 3′ end, (i) causes extension from the new 3′ end, (ii) displaces the X domain to form the single-stranded EXPAR trigger X, and (iii) reforms the first double-stranded fragment; repeated nicking, strand extension, and strand displacement at the first double-stranded fragment linearly amplifies the EXPAR trigger X; primers Ay, By, and Cy, when exposed to the Ply, P2y, and P3y domains of the second target sequence, trigger a series of reactions that enable the polymerase to form the second double-stranded fragment, wherein a strand of the second double-stranded fragment comprises, in the 5′ to 3′ direction, domains P3y, Ply, R, and Y; the nicking enzyme, when exposed to the second double-stranded fragment, nicks within the R domain to form a new 3′ end upstream of the Y domain; the polymerase, when exposed to the new 3′ end, (i) causes extension from the new 3′ end, (ii) displaces the Y domain to form the single-stranded EXPAR trigger Y, and (iii) reforms the second double-stranded fragment; and repeated nicking, strand extension, and strand displacement at the second double-stranded fragment linearly amplifies the EXPAR trigger Y.

Clause 31. The composition of clause 29 or 30, wherein the EXPAR template comprises: (a) in the 5′ to 3′ direction, (i) an X′ domain or a Z′ domain, (ii) an R′ domain, (iii) a Y′ domain, (iv) a loop-forming domain L optionally flanked by extended hairpin stem domains E and E′, (v) a Y domain, and (vi) an X′ domain, wherein the Y′ and Y domains hybridize to form a stem portion and a loop, wherein hybridization of EXPAR trigger X to the 3′ most X′ domain and extension therefrom causes displacement of the Y domain from the Y′ domain to open the stem and thereby enable hybridization of EXPAR trigger Y to the exposed Y′ domain and extension therefrom to linearly amplify additional first EXPAR trigger X or third EXPAR trigger Z sequences via repeated cycles of nicking within the R domain to form a new 3′ end, strand extension from the new 3′ end, and strand displacement of the downstream X or Z domain, or (b) in the 5′ to 3′ direction, (i) a Y′ domain or a Z′ domain, (ii) an R′ domain, (iii) an X′ domain, (iv) a hairpin loop domain L optionally flanked by extended hairpin stem domains E and E′, (v) an X domain, and (vi) a Y′ domain, wherein the X′ and X domains hybridize to form a stem portion and a loop, wherein hybridization of EXPAR trigger Y to the 3′ most Y′ domain and extension therefrom causes displacement of the X domain from the X′ domain to open the stem and thereby enable hybridization of EXPAR trigger X to the exposed X′ domain and extension therefrom to linearly amplify additional second EXPAR trigger Y or third EXPAR trigger Z sequences via repeated cycles of nicking within the R domain to form a new 3′ end, strand extension from the new 3′ end, and strand displacement of the downstream Y or Z domain, wherein the linearly amplified sequences function as additional EXPAR trigger X or EXPAR trigger Y when the 3′ most domain on the EXPAR template is an X′ or a Y′ domain, respectively, and are available to subsequently react with additional EXPAR templates to enable exponential amplification.

Clause 32. The composition of any of clauses 29-31, wherein the EXPAR template comprises an extension stopper to prevent the polymerase from driving extension into the loop-forming domain L.

Clause 33. The composition of clause 32, wherein the extension stopper comprises an extension stopping base pair, such as an iso-dG-iso-dC base pair, each nucleotide flanking opposite sides of the hairpin loop domain L or being disposed adjacent stem domains E and E′.

Clause 34. The composition of any of clauses 29-33, wherein the first primer set, second primer set, or both include one or more features of the composition of any of claims 1-28.

Clause 35. A composition formulated to enable detection of first or second target nucleic acid sequences through isothermal amplification when either the first or second target sequences are present within a sample, the composition comprising: (1) a first primer set comprising a primer Ax comprising, in the 5′ to 3′ direction, an X′ domain that is reverse complementary to a first EXPAR trigger X, an R′ domain that is reverse complementary to a nicking enzyme recognition site R, and a P1x′ domain that is reverse complementary to a P1x domain of the first target sequence, wherein the X′ and R′ domains are independent from the first target sequence; a primer Bx comprising a P2x′ domain that is reverse complementary to a P2x domain of the first target sequence, wherein P2x is located 3′ of P1x in the first target sequence; a primer Cx comprising a P3x domain that is equivalent to a P3x domain of the first target sequence, wherein P3x is located 5′ of P1x in the first target sequence, wherein the primers Ax, Bx, and Cx, when exposed to the P1x, P2x, and P3x domains of the target sequence, trigger a series of reactions that result in a first double-stranded fragment used to linearly amplify the EXPAR trigger X via repeated cycles of nicking, strand extension, and strand displacement; (2) a second primer set comprising a primer Ay comprising, in the 5′ to 3′ direction, a Y′ domain that is reverse complementary to a second EXPAR trigger Y, an R′ domain that is reverse complementary to a nicking enzyme recognition site R, and a Ply′ domain that is reverse complementary to a Ply domain of the second target sequence, wherein the Y′ and R′ domains are independent from the second target sequence; a primer By comprising a P2y′ domain that is reverse complementary to a P2y domain of the second target sequence, wherein P2y is located 3′ of Ply in the second target sequence; a primer Cy comprising a P3y domain that is equivalent to a P3y domain of the second target sequence, wherein P3y is located 5′ of Ply in the second target sequence; wherein the primers Ay, By, and Cy, when exposed to the Ply, P2y, and P3y domains of the target sequence, trigger a series of reactions that result in a second double-stranded fragment used to linearly amplify the EXPAR trigger Y via repeated cycles of nicking, strand extension, and strand displacement; (3) a polymerase enzyme that exhibits strand-displacing functionality; (4) a nicking enzyme that recognizes the nicking enzyme recognition site R; and (5) a first EXPAR template configured to generate additional EXPAR trigger Y upon exposure to the EXPAR trigger X; and (6) a second EXPAR template configured to generate additional EXPAR trigger X upon exposure to the EXPAR trigger Y.

Clause 36. The composition of clause 35, wherein: primers Ax, Bx, and Cx, when exposed to the P1x, P2x, and P3x domains of the first target sequence, trigger a series of reactions that enable the polymerase to form the first double-stranded fragment, wherein a strand of the first double-stranded fragment comprises, in the 5′ to 3′ direction, domains P3x, P1x, R, and X; the nicking enzyme, when exposed to the first double-stranded fragment, nicks within the R domain to form a new 3′ end upstream of the X domain; the polymerase, when exposed to the new 3′ end, (i) causes extension from the new 3′ end, (ii) displaces the X domain to form the single-stranded EXPAR trigger X, and (iii) reforms the first double-stranded fragment; repeated nicking, strand extension, and strand displacement at the first double-stranded fragment linearly amplifies the EXPAR trigger X; primers Ay, By, and Cy, when exposed to the Ply, P2y, and P3y domains of the second target sequence, trigger a series of reactions that enable the polymerase to form the second double-stranded fragment, wherein a strand of the second double-stranded fragment comprises, in the 5′ to 3′ direction, domains P3y, Ply, R, and Y; the nicking enzyme, when exposed to the second double-stranded fragment, nicks within the R domain to form a new 3′ end upstream of the Y domain; the polymerase, when exposed to the new 3′ end, (i) causes extension from the new 3′ end, (ii) displaces the Y domain to form the single-stranded EXPAR trigger Y, and (iii) reforms the second double-stranded fragment; and repeated nicking, strand extension, and strand displacement at the second double-stranded fragment linearly amplifies the EXPAR trigger Y.

Clause 37. The composition of clause 35 or 36, wherein: (a) the first EXPAR template comprises, in the 5′ to 3′ direction, (i) a Y domain, (ii) a loop-forming domain L optionally flanked by extended hairpin stem domains E and E′, (iii) a Y′ domain, (iv) an R′ domain, and (v) an X′ domain, wherein the Y′ and Y domains hybridize to form a stem portion and a loop, wherein hybridization of EXPAR trigger X to the X′ domain and extension therefrom causes displacement of the Y domain from the Y′ domain to open the stem and thereby enable generation of additional EXPAR trigger Y via repeated cycles of nicking, strand extension, and strand displacement; and/or (b) the second EXPAR template comprises, in the 5′ to 3′ direction, (i) an X domain, (ii) a loop-forming domain L optionally flanked by extended hairpin stem domains E and E′, (iii) an X′ domain, (iv) an R′ domain, and (v) a Y′ domain, wherein the X′ and X domains hybridize to form a stem portion and a loop, wherein the hybridization of EXPAR trigger Y to the Y′ domain and extension therefrom causes displacement of the X domain from the X′ domain to open the stem and thereby enable generation of additional EXPAR trigger X via repeated cycles of nicking, strand extension, and strand displacement.

Clause 38. The composition of any of clauses 35-37, wherein the first and/or second EXPAR templates comprise an extension stopper to prevent the polymerase from driving extension into the loop-forming domain L.

Clause 39. The composition of clause 38, wherein the extension stopper comprises an extension stopping base pair, such as an iso-dG-iso-dC base pair, each nucleotide flanking opposite sides of the hairpin loop domain L or being disposed adjacent stem domains E and E′.

Clause 40. The composition of any of clauses 35-39, wherein the first and/or second EXPAR templates comprise an extension stopper at the 3′ end, such as a 3′ Inverted dT, 3′ Phosphorylation, 3′ Amino Modifier C6 dT, poly-T tail, or other 3′ modifications that block polymerase extension.

Clause 41. The composition of any of clauses 35-40, wherein the first primer set, second primer set, or both include one or more features of the composition of any of claims 1-28.

Clause 42. A method for carrying out an isothermal amplification reaction for detection of a target nucleic acid sequence, the method comprising: mixing a reaction mixture with a sample; in a first amplification stage, performing a transduction reaction to generate and linearly amplify a single-stranded oligonucleotide when the target sequence is present in the sample, wherein the single-stranded oligonucleotide is configured to function as an EXPAR trigger sequence for initiating a downstream EXPAR amplification reaction; and in a second amplification stage, performing the EXPAR amplification reaction to generate additional EXPAR trigger sequences.

Clause 43. The method of clause 42, wherein the reaction mixture comprises a composition as in any of claims 1-41.

Clause 44. The method of any of clauses 42-43, wherein the target nucleic acid is DNA.

Clause 45. The method of any of clauses 42-43, wherein the target nucleic acid is RNA, and wherein the first amplification stage is carried out using a strand-displacing polymerase with reverse transcriptase activity or a strand-displacing polymerase with an additional reverse transcriptase.

Clause 46. The method of any of clauses 42-45, wherein the target sequence is independent of sequences used in the second amplification stage and the second amplification stage does not require amplification of any portion of the target sequence.

Clause 47. The method of any of clauses 42-46, wherein the EXPAR trigger sequence generated in the first amplification stage is the same as the additional EXPAR trigger sequences generated in the second amplification stage.

Clause 48. The method of any of clauses 42-47, wherein the second amplification stage provides exponential amplification of the EXPAR trigger sequence.

Clause 49. The method of any of clauses 42-46, wherein the EXPAR trigger sequence generated in the first amplification stage is different than the additional EXPAR trigger sequences generated in the second amplification stage.

Clause 50. The method of any of clauses 42-49, wherein at least a portion of the first amplification stage and at least a portion of the second amplification stage occur concurrently.

Clause 51. The method of any of clauses 42-50, wherein the first and second amplification stages are carried out in a one-pot format in the same reaction mixture.

### Additional Terms & Definitions

As used herein, domains that are “adjacent” to one another omit other functional domains therebetween, “Adjacent domains” can include one or more intervening nucleotides (e.g., 2 to 50). Accordingly, a first domain and a second domain are “adjacent domains” if there are no other functional domains disposed therebetween but are not necessarily directly adjacent. A functional domain is one that is involved in the amplification reaction by enabling primer hybridization, enzyme recognition, or EXPAR trigger hybridization, for example, and can include primer binding sites, loop-forming regions, and catalytic recognition sites.

A domain that is “independent from the target sequence” means a domain wherein neither the sequence of the domain nor its reverse complement is included in the target sequence.

While certain embodiments of the present disclosure have been described in detail, with reference to specific configurations, parameters, components, elements, etcetera, the descriptions are illustrative and are not to be construed as limiting the scope of the claimed invention.

For any given element of component of a described embodiment, any of the possible alternatives listed for that element or component may be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. Each numerical parameter should be construed in light of the number of reported significant digits and by applying ordinary rounding techniques.

Any headings and subheadings used herein are for organizational purposes only and are not meant to be used to limit the scope of the description or the claims.

As used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, isothermal amplification components such as enzymes, primers, and detection probes that are not specifically disclosed herein may optionally be excluded.

### REFERENCES

(1) Oliveira, B. B.; Veigas, B.; Baptista, P. V. Isothermal Amplification of Nucleic Acids: The Race for the Next “Gold Standard.” Front. Sens. 2021, 2.
(2) Notomi, T.; Okayama, H.; Masubuchi, H.; Yonckawa, T.; Watanabe, K.; Amino, N.; Hase, T. Loop-Mediated Isothermal Amplification of DNA. Nucleic Acids Res. 2000, 28 (12), e63.
(3) Li, J.; Macdonald, J.; Stetten, F. von. Review: A Comprehensive Summary of a Decade Development of the Recombinase Polymerase Amplification. Analyst 2018, 144 (1), 31-67.
(4) Van Ness, J.; Van Ness, L. K.; Galas, D. J. Isothermal Reactions for the Amplification of Oligonucleotides. Proc. Natl. Acad. Sci. 2003, 100 (8), 4504-4509.
(5) Reid, M. S.; Le, X. C.; Zhang, H. Exponential Isothermal Amplification of Nucleic Acids and Assays for Proteins, Cells, Small Molecules, and Enzyme Activities: An EXPAR Example. Angew. Chem. Int. Ed. 2018, 57 (37), 11856-11866.
(6) Tan, E.; Wong, J.; Nguyen, D.; Zhang, Y.; Erwin, B.; Van Ness, L. K.; Baker, S. M.; Galas, D. J.; Niemz, A. Isothermal DNA Amplification Coupled with DNA Nanosphere-Based Colorimetric Detection. Anal. Chem. 2005, 77 (24), 7984-7992.
(7) Tan, E.; Erwin, B.; Dames, S.; Ferguson, T.; Buechel, M.; Irvine, B.; Voelkerding, K.; Niemz, A. Specific versus Nonspecific Isothermal DNA Amplification through Thermophilic Polymerase and Nicking Enzyme Activities. Biochemistry 2008, 47 (38), 9987-9999.
(8) Urtel, G.; Van Der Hofstadt, M.; Galas, J. C.; Estevez-Torres, A. rEXPAR: An Isothermal Amplification Scheme That Is Robust to Autocatalytic Parasites. Biochemistry 2019, 58 (23), 2675-2681.
(9) Reid, M. S.; Paliwoda, R. E.; Zhang, H.; Lc, X. C. Reduction of Background Generated from Template-Template Hybridizations in the Exponential Amplification Reaction. Anal. Chem. 2018, 90 (18), 11033-11039.
(10) Qian, J.; Ferguson, T. M.; Shinde, D. N.; Ramírez-Borrero, A. J.; Hintze, A.; Adami, C.; Niemz, A. Sequence Dependence of Isothermal DNA Amplification via EXPAR. Nucleic Acids Res. 2012, 40 (11), c87-c87.
(11) Tan, E.; Erwin, B.; Dames, S.; Voelkerding, K.; Niemz, A. Isothermal DNA Amplification with Gold Nanosphere-Based Visual Colorimetric Readout for Herpes Simplex Virus Detection. Clin. Chem. 2007, 53 (11), 2017-2020.
(12) Yan, Y.; Zhao, D.; Yuan, T.; Hu, J.; Zhang, D.; Cheng, W.; Zhang, W.; Ding, S. A Simple and Highly Sensitive Electrochemical Biosensor for microRNA Detection Using Target-Assisted Isothermal Exponential Amplification Reaction. Electroanalysis 2013, 25 (10), 2354-2359.
(13) Song, J.; Kim, S.; Kim, H. Y.; Hur, K. H.; Kim, Y.; Park, H. G. A Novel Method to Detect Mutation in DNA by Utilizing Exponential Amplification Reaction Triggered by the CRISPR-Cas9 System. Nanoscale 2021, 13 (15), 7193-7201.
(14) Liu, H.; Tian, T.; Zhang, Y.; Ding, L.; Yu, J.; Yan, M. Sensitive and Rapid Detection of microRNAs Using Hairpin Probes-Mediated Exponential Isothermal Amplification. Biosens. Bioelectron. 2017, 89, 710-714.
(15) Lcc, S.; Jang, H.; Kim, H. Y.; Park, H. G. Three-Way Junction-Induced Isothermal Amplification for Nucleic Acid Detection. Biosens. Bioelectron. 2020, 147, 111762.
(16) Abrosimova, L. A.; Kisil, O. V.; Romanova, E. A.; Oretskaya, T. S.; Kubareva, E. A. Nicking Endonucleases as Unique Tools for Biotechnology and Gene Engineering. Russ. J. Bioorganic Chem. 2019, 45 (5), 303-320.
(17) Cao, S.; Tang, X.; Chen, T.; Chen, G. Types and Applications of Nicking Enzyme-Combined Isothermal Amplification. Int. J. Mol. Sci. 2022, 23 (9), 4620.
(18) Qian, C.; Wang, R.; Wu, H.; Ji, F.; Wu, J. Nicking Enzyme-Assisted Amplification (NEAA) Technology and Its Applications: A Review. Anal. Chim. Acta 2019, 1050, 1-15.
(19) Zhang, D. Y. Towards Domain-Based Sequence Design for DNA Strand Displacement Reactions. In DNA Computing and Molecular Programming; Sakakibara, Y., Mi, Y., Eds.; Lecture Notes in Computer Science; Springer: Berlin, Heidelberg, 2011; pp 162-175.

1. A composition formulated to enable detection of a target nucleic acid sequence using isothermal amplification, the composition comprising:
a primer A comprising, in the 5′ to 3′ direction, an X′ domain, an R′ domain that is reverse complementary to a nicking enzyme recognition site R, and a P1′ domain that is reverse complementary to a P1 domain of the target sequence, wherein the X′ and R′ domains are independent from the target sequence;
a primer B comprising a P2′ domain that is reverse complementary to a P2 domain of the target sequence, wherein P2 is located 3′ of P1 in the target sequence;
a primer C comprising a P3 domain that is equivalent to a P3 domain of the target sequence, wherein P3 is located 5′ of P1 in the target sequence;
a polymerase enzyme that exhibits strand-displacing functionality;
a nicking enzyme that recognizes a double-stranded site of hybridized R and R′ domains and nicks the R domain; and
an EXPAR template,
wherein primers A, B, and C, when exposed to the P1, P2, and P3 domains of the target sequence, trigger a series of reactions that result in a double-stranded fragment used to carry out a first amplification stage that linearly amplifies an EXPAR trigger X via repeated cycles of nicking, strand extension, and strand displacement, and
wherein the EXPAR template is configured to hybridize with the EXPAR trigger X to initiate a second amplification stage that exponentially amplifies the EXPAR trigger X via repeated cycles of nicking, strand extension, and strand displacement.

2. The composition of claim 1, wherein the composition is formulated such that:
primers A, B, and C, when exposed to the P1, P2, and P3 domains of the target sequence, trigger a series of reactions that enable the polymerase to form the double-stranded fragment, wherein a first strand of the double-stranded fragment comprises, in the 5′ to 3′ direction, domains P3, P1, R, and X;
the nicking enzyme, when exposed to the double-stranded fragment, nicks within the R domain of the first strand to form a new 3′ end upstream of the X domain;
the polymerase, when exposed to the new 3′ end, (i) extends the new 3′ end, (ii) displaces the X domain to form the single-stranded EXPAR trigger X, and (iii) reforms the double-stranded fragment; and
repeated nicking, strand extension, and strand displacement at the double-stranded fragment define the first amplification stage that linearly amplifies the EXPAR trigger X.

3. The composition of claim 1, wherein the EXPAR template comprises, in the 3′ to 5′ direction, a first (3′ most) X′ domain, an R′ domain, and a second (5′ most) X′ domain.

4. The composition of claim 1, wherein excess primer A functions as EXPAR template, and further comprises an additional R′ domain located 5′ of the X′ domain and an additional X′ domain located 5′ of the additional R′ domain such that following the series of reactions that enable formation of the double-stranded fragment, a first strand of the double-stranded fragment comprises, in the 5′ to 3′ direction, domains P3, P1, 5′ most R, 5′ most X, 3′ most R, and 3′ most X.

5. The composition of claim 1, wherein the primer A further comprises an S′ domain, located 5′ of the R′ domain and 3′ of the X′ domain, that is reverse complementary to a stabilization domain S, and further comprises a strand that comprises S and X domains and is hybridized to the S′ and X′ domains to form a gated primer such that following the series of reactions that enable formation of the double-stranded fragment, a first strand of the double-stranded fragment comprises, in the 5′ to 3′ direction, domains P3, P1, R, S, and X.

6. The composition of claim 1, further comprising an initiator D, wherein the primer A further comprises a D′ domain, located 5′ of the R′ domain and 3′ of the X′ domain, that is reverse complementary to the initiator D such that when initiator D is hybridized to domain D′ of primer A, the initiator D is extended to form a strand that comprises D and X domains hybridized to the D′ and X′ domains of the primer A to form a gated primer.

7. The composition of claim 3, wherein the EXPAR template is configured to form a hairpin loop that limits hybridization of the EXPAR trigger X to the 5′ most X′ domain of the EXPAR template, wherein the EXPAR template comprises a loop-forming domain L located 5′ of the 5′ most X′ domain and an X or partial X domain located 5′ of the loop-forming domain to enable formation of the hairpin loop.

8. The composition of claim 7, wherein the EXPAR template further comprises an extended hairpin stem domain E located 5′ of the L domain and an E′ domain that is reverse complementary to the E domain and is located 3′ of the L domain, the E and E′ domains configured to form an extended hairpin stem.

9. The composition of claim 7, wherein the EXPAR template further comprises an extension stopper to prevent the polymerase from driving extension into the loop-forming domain L when the EXPAR trigger X hybridizes to the 3′ most X′ domain of the EXPAR template and is extended therefrom.

10. The composition of claim 9, wherein the extension stopper comprises an extension stopping base pair.

11. The composition of claim 10, wherein the extension stopping base pair comprises an iso-dG-iso-dC base pair.

12. The composition of claim 1, further comprising a labeled probe comprising an X′ domain for hybridization with the EXPAR trigger X, the labelled probe being configured to generate a fluorescence signal in response to interaction with the EXPAR trigger X.

13. A method for carrying out an isothermal amplification reaction for detection of a target nucleic acid sequence, the method comprising:
mixing a reaction mixture with a sample;
in a first amplification stage, performing a transduction reaction to generate and linearly amplify a single-stranded oligonucleotide when the target sequence is present in the sample, wherein the single-stranded oligonucleotide is configured to function as an EXPAR trigger sequence for initiating a downstream EXPAR amplification reaction; and
in a second amplification stage, performing the EXPAR amplification reaction to generate additional EXPAR trigger sequences.

14. The method of claim 13, wherein the reaction mixture comprises a composition that includes:
a primer A comprising, in the 5′ to 3′ direction, an X′ domain, an R′ domain that is reverse complementary to a nicking enzyme recognition site R, and a P1′ domain that is reverse complementary to a P1 domain of the target sequence, wherein the X′ and R′ domains are independent from the target sequence;
a primer B comprising a P2′ domain that is reverse complementary to a P2 domain of the target sequence, wherein P2 is located 3′ of P1 in the target sequence;
a primer C comprising a P3 domain that is equivalent to a P3 domain of the target sequence, wherein P3 is located 5′ of P1 in the target sequence;
a polymerase enzyme that exhibits strand-displacing functionality;
a nicking enzyme that recognizes a double-stranded site of hybridized R and R′ domains and nicks the R domain; and
an EXPAR template,
wherein primers A, B, and C, when exposed to the P1, P2, and P3 domains of the target sequence, trigger a series of reactions that result in a double-stranded fragment used to carry out a first amplification stage that linearly amplifies an EXPAR trigger X via repeated cycles of nicking, strand extension, and strand displacement, and
wherein the EXPAR template is configured to hybridize with the EXPAR trigger X to initiate a second amplification stage that exponentially amplifies the EXPAR trigger X via repeated cycles of nicking, strand extension, and strand displacement.

15. The method of claim 13, wherein the target sequence is independent of sequences used in the second amplification stage and the second amplification stage does not require amplification of any portion of the target sequence.

16. The method of claim 13, wherein the EXPAR trigger sequence generated in the first amplification stage is the same as the additional EXPAR trigger sequences generated in the second amplification stage.

17. The method of claim 13, wherein the second amplification stage provides exponential amplification of the EXPAR trigger sequence.

18. The method of claim 13, wherein the EXPAR trigger sequence generated in the first amplification stage is different than the additional EXPAR trigger sequences generated in the second amplification stage.

19. The method of claim 13, wherein at least a portion of the first amplification stage and at least a portion of the second amplification stage occur concurrently.

20. The method of claim 13, wherein the first and second amplification stages are carried out in a one-pot format in the same reaction mixture.
